(19) United States
(12) Patent Application Publication
(10) Pub. No.: US 2026/0215896 A1
(43) Pub. Date: Jul. 30, 2026

(54) AN INTRAORAL SCANNING SYSTEM FOR DETERMINING COMPOSED SCAN INFORMATION

(71) Applicant: 3SHAPE A/S, Copenhagen K (DK)

(72) Inventors: Philip Grabow WESTERGAARD, Copenhagen K (DK); Christoph VANNAHME, Copenhagen K (DK); Stavroula MICHOU, Copenhagen K (DK); Alexander Bruun CHRISTIANSEN, Copenhagen K (DK)

(73) Assignee: 3SHAPE A/S, Copenhagen K (DK)

(21) Appl. No.: 19/145,077

(22) PCT Filed: Dec. 19, 2023
(86) PCT No.: PCT/EP2023/086744

§ 371 Date: Jul. 1, 2025

(30) Foreign Application Priority Data
Jan. 3, 2023 (DK) PA202370003

(51) Int. Cl. A61C 9/00; A61C 19/04

(52) U.S. Cl. CPC A61C 9/0053; A61C 19/04

(57) ABSTRACT

An intraoral scanning system includes a handheld intraoral scanning device that includes an image sensor unit configured to capture visible light information and internal light information from at least the dental arch caused by the emitted light at the different wavelengths. The system includes one or more processors operably connected to the image sensor unit, the one or more processors is configured to receive the visible light information and the internal light information, determine, in real time, surface information from the visible light information for generating or updating a three-dimensional (3D) model of the dental arch using the surface information, determine internal structure information of the dental arch from the internal light information, and determine composed scan information including a composition of the internal structure information and the visible light information.

### FIELD

The disclosure relates to an intraoral scanning system. More specifically, the disclosure relates to one or more processors of the system that is configured to determine a composed scan information that includes enhanced internal structure information and/or enhanced textural information of a tooth.

### BACKGROUND

Many dental and orthodontic procedures can benefit from accurate three-dimensional (3D) descriptions of a patient's dentition and intraoral cavity. In particular, it would be helpful to provide a three-dimensional description of both the surface and internal structures of the teeth, including the enamel and dentin, as well as caries and the general internal composition of the tooth volume. Although pure surface representations of the 3D surfaces of teeth have proven extremely useful in the design and fabrication of dental restorations (e.g., crowns or bridges) the ability to image internal structures including the development of caries and dental cracks in the enamel and underlying dentin would be tremendously useful, particularly in conjunction with a surface topographical mapping.

State of the art, ionizing radiation (e.g., X-rays) has been used to image the teeth for diagnostic purposes. For example, X-ray bitewing radiographs are often used to provide non-quantitative images of the teeth's internal structures. However, in addition to the risk of ionizing radiation, such images are typically limited in their ability to show early tooth mineralization changes (e.g. initial caries) resulting in underestimation of the demineralization depth; they are unable to assess the presence or not of micro-cavitation; they result in frequent overlap of the approximal tooth surfaces which requires repetition of radiograph acquisition and thus may involve a lengthy and expensive procedure. Some intraoral features such as soft tissues and dental plaque are usually not visualized via X-ray because of their low density. Other techniques, such as cone beam computed tomography (CBCT) may provide tomographic images and be used to collect more information about the tissues and internal structure, but still require ionizing radiation.

Furthermore, it is known that near-infrared (NIR) light can be used for assessing internal structure of a tooth and tooth surface in the form of transillumination of teeth or light reflection and backscattering from teeth. The NIR range offers a non-ionizing and safe approach to assess dental caries, restorations, cracks, enamel and dentin defects.

### SUMMARY

It is an aspect of the present disclosure to improve the detection of internal structures using a non-ionizing and sage approach.

A further aspect of the present disclosure is to obtain an intraoral scanning system that is configured to enhance the visualization of caries of teeth in such a manner that it becomes easier for the dental to identify and treat caries.

Another aspect of the present disclosure is to obtain an intraoral scanning system that is configured to enhance the visualization of restorations of teeth in such a manner that it becomes easier for the dental to identify and treat second order caries, and, to identify the type of restoration, such as type of fillings, inlays, onlays, crowns and/or sealants.

Yet another aspect of the present disclosure is to obtain an intraoral scanning system that is configured to enhance the visualization of “dentin-enamel junction” (DEJ) of teeth in such a manner that it becomes easier for the dentist to identify the dentin-enamel junction for clinical assessment.

A further aspect of the present disclosure is to obtain an intraoral scanning system that is configured to generate or update a three-dimensional 3D model while determine composed scan information with use of different wavelength modalities.

Besides being able to enhance the visualization of caries, restoration, and dentin-enamel junction, yet a further aspect of the present disclosure is to detect incipient caries at stages where preventive measures are likely to effect remineralization, repairing damage done by the caries infection at a stage well before more complex restorative measures are necessary. Advantageously, the disclosure can be accurate at an earlier stage of caries infection than has been exhibited using existing fluorescence or near-infrared approaches. According to the aspects, an intraoral scanning system is disclosed. The intraoral scanning system may be configured to provide a composed scan data based on captured visible light information and internal light information. The system may include a handheld intraoral scanning device that includes a projector unit configured to emit light with different wavelengths during time periods onto at least a dental arch, wherein the different wavelengths include at least a near-infrared wavelength and at least a visible wavelength. The wavelength of each time periods may be different. The handheld intraoral scanning device may further include an image sensor unit configured to capture visible light information and internal light information from at least the dental arch caused by the emitted light at the different wavelengths. The system may further comprise one or more processors operably connected to the image sensor unit, the one or more processors may be configured to receive the visible light information and the internal light information, and to determine, in real time, surface information from the visible light information for generating or updating a three-dimensional (3D) model of the dental arch using the surface information. The one or more processors may be configured to determine internal structure information of the dental arch from the internal light information, and to determine composed scan information including a composition of the internal structure information and the visible light information.

The internal light information includes mainly reflection from inside a tooth, i.e. internal region of a dental object, and significantly less of surface reflection of a tooth. The visible light information includes mainly surface reflection of a tooth and significantly less reflection from inside a tooth, i.e. internal region of a dental object. The internal light information includes information about dental condition from within a tooth and not just surficial.

A composed scan information does not include an overlay of two 2D images where each of the two 3D images relates to different emitted wavelengths from the projector unit. In this example, no enhancement of internal structure information is provided. The composed scan information may be a combination of intensity levels of each pixel of the image sensor unit that relates to different wavelengths. For example, at a first time period, the image sensor unit captures light information that relates to visible wavelength, and at a second time period, the image sensor unit captures light information that relates to a near-infrared wavelength, and the intensity levels of the two time periods are recorded and combined for enhancing a dental condition, i.e. an internal structure information. The combination of the intensity levels may be done digitally by subtraction and/or addition of the intensity levels. In another example, the intensity levels may be captured and recorded during at least three time periods for at least three different wavelengths, such as white-coloured wavelength, blue-coloured wavelength and near-infrared wavelength.

The one or more processors may be configured to display the composed scan information and the 3D model on a displaying unit of the system.

The one or more processors may be configured to utilize the visible light information for both generating or updating the 3D model and for determining the composed scan information that includes enhanced internal structures in the form of enhanced contrast between the internal structure, such as a caries lesion, and tooth structures. The tooth structures are mainly provided by the visible light information and the internal structures are mainly provided by the internal structure information. The utilization of the visible light information for both the 3D model and the composed scan information results in an easier mapping of the composed scan information onto the 3D model or to determine a location of the composed scan information on the 3D model.

In another example where a handheld intraoral scanning device first performs a scan for generating or updating a 3D model and then afterwards performs another scan for determining composed scan information would result in a more complicated way of aligning the position of the composed scan information on the 3D model in comparison to the present disclosure.

In another example, the intraoral scanning system may perform a scanning sequence where the visible light information is received during a first time period, and the received visible light information is dedicated only for generating or updating a 3D model, and then in a subsequent time period, the new received visible light information is dedicated only for determining composed scan information. This example would result in a lower scanning rate for obtaining a similar quality for the 3D model as if the visible light information received during the first time period would be used for also determining the composed scan information.

The present disclosure allows a faster scanning rate for providing both scan information for the 3D model and for the composed scan information, because the scan information relevant for generating or updating the 3D model are also being used for determining the composed scan information.

The composed scan information includes enhanced internal structure information in comparison to near-infrared information and fluorescence information, solely, or in comparison to enhanced fluorescence information where specular reflection has been removed. Internal structures of a tooth are more easily identified in near-infrared information than in fluorescence information, as the contrast between the internal structures and surface structures is higher for the near-infrared information. Therefore, it would not be of any interest in trying to enhance internal structure information in fluorescence information. In the present disclosure, the composed scan information includes enhanced internal structure information. The enhanced internal structure information includes enhanced contrast between the internal structures and surface structures of a tooth. Thereby, it would be easier for a dental to identify incipient caries at stages where preventive measures are likely to effect remineralization, repairing damage done by the caries infection at a stage well before more complex restorative measures are necessary. Furthermore, if using fluorescence information to subtract, a demineralization is always dark and thus the IR signal gets enhanced. If you use a white light image an early caries lesion is bright (white spot) but an older lesion is dark. Thus, this cannot consistently enhance the IR signal as well as a fluo image.

I would also add that the structural similarity between Fluo and NIR images (not present in the visible image) makes for a nice common background to be subtracted, thereby highlighting the features that we are interested in.

(not by increasing the signal, but by reducing the background)

The one or more processors may be configured to determine fluorescence information of the dental arch from the visible light information. The fluorescence information may include a green fluorescence information and/or red fluorescence information. In this example, the projector unit may include multiple light sources that are configured to emit light that is ideal for determining surface information, fluorescence information and internal structure information of teeth of the dental arch.

The composed scan information may include a difference between the internal structure information and the visible light information, or between the internal structure information and the fluorescence information. The differences result in enhanced internal structure information in relation to a near-infrared image, a fluorescence image or an enhanced fluorescence image. Furthermore, the difference between the internal structure information and the fluorescence information results in a further enhanced internal structure information in comparison to a composed scan information that includes the difference between the internal structure information and surface information.

The one or more processors may be configured to determine a first difference between the internal structure information and the green fluorescence information and a second difference between the internal structure information and the red fluorescence information, and wherein the one or more processors may be configured to determine the composed scan information by a summation of the first difference and the second difference. The summation of the two composed scan information has shown to be ideal for enhancing internal structure information that relates to specifically caries lesion, and thereby, easier to distinguish from other structural information of the tooth.

The composed scan information may include a summation of the internal structure information, the green fluorescence information and the red fluorescence information, and in relation to the previous example where two composed scan information have been added together, dentin-enamel junction is more enhanced, and thereby, easier to distinguish from other structural information of the tooth.

The composed scan information may include a difference between the internal structure information and the surface information, or vice versa. In this example, the composed scan information is ideal for enhancing internal structure information that relates to restoration.

It has been shown that the order of the internal structure information and the surface information is relevant for enhancing restorations of different type. For example, the composed scan information including a subtraction of the internal structure information with the surface information is ideal for enhancing internal structural information that relates to a first type of restorations, and, the composed scan information including the a subtraction of the surface information with the internal structure information is ideal for enhancing internal structure information that relates to a second type of restorations. lor The one or more processors may be configured to determine the composed scan information such that the composition of the internal structure information and the visible light information is based on light intensity from the image sensor unit that corresponds to the internal structure information and the visible light information. In another example, the one or more processors may be configured to determine the composed scan information such that the composition of the internal structure information and the visible light information implies merging of the light intensities of the internal structure information and the visible light information, where each of the two images corresponds to the internal structure information and the visible light information, respectively. The merging of the light intensities may imply adding, subtracting, weighting of the light intensities.

The composition may correspond to a light intensity difference between light intensity of the internal structure information and light intensity of the visible light information, or vice versa, or a light intensity summation between light intensity of the internal structure information and light intensity of the visible light information.

The composition may include a light intensity difference between light intensity of the internal structure information and light intensity of the visible light information, wherein the intensity difference enhances the internal structure information. The composition may be applied on to the 3D model or a two-dimensional image that includes visible light information, such as white information that corresponds to surface information of the dental object.

The composition of the internal structure information and the visible light information does not imply adding, combining or merging two images together for the purpose of displaying or visualize two images at the same time. Furthermore, the composition of the internal structure information and the visible light information does not imply adding, combining or merging two images together for the purpose of displaying or visualize two images on top of each other. The purpose of composition is to enhance the internal structure information of the dental object/arch. By enhancing the internal structure information means that the visibility of the internal structure information is improved significantly in comparison to internal structure information identified only by an infrared image.

The light intensity may correspond to a light intensity of one or more pixels of the image unit, and in the composed scan information, an area of pixels of the image sensor unit configured to capture the information corresponds between the captured visible light information and the internal light information. The composed scan information may be in the form of a bitmap suitable for being overlayed on a 2D image or a 3D model or a 2D image, such as a 256×256 image, where each pixels may be generated from subtracting and/or addition of two or more different 2D images, i.e. light information captured by different wavelengths.

The visible light information may be predominated by surface information, and a subtraction of the visible light information with the internal structure information would result in a composed scan information with less surface information in the internal structure information. The one or more processors may be configured to change the impact of the visible light information on the internal structure information by applying a weighting coefficient to each of the information, wherein a decrease of the weighting coefficient of the visible light information would correspond to less removal of surface information from the internal structure information, or vice versa, a larger weighting of the visible light information would result in more removal of surface information from the internal structure information.

The internal structure information and the visible light information may be weighted in the composed scan information, and wherein the internal structure information is weighted by a first weighting coefficient and the visible light information is weighted by a second weighting coefficient. When determining the composed scan information, the visible light information and the internal structure information may contribute relatively differently. The relative contribution from each of the information may be controlled by the weighting coefficients. The visible light information may include both surface information and fluorescence information, and in this example, the relative contribution of the surface information may be weighted by a first primary weighting coefficient, and the relative contribution of the fluorescence information may be weighted by a first secondary weighting coefficient.

The one or more processors may be configured to change the first and the second weighting coefficient, and wherein the change may correspond to a change in brightness, contrast and/or transparency of the internal structure information and the visible light information, respectively. Different levels of the weighting coefficients may be ideal for different dental conditions to be enhanced, e.g. an enhanced internal structure and/or enhanced textural information. For example, if the composed scan information includes a difference between the internal structure information and the visible light information, and the weighting coefficient for the visible light information is smaller than for the internal structure information, the composed scan information would be ideal for detecting restorations, and a larger weighting coefficient for the visible light information would result in a composed scan information that are ideal for detecting caries. Thereby, the one or more processors may be configured to automatically determine the level of the weighting coefficients based on a dental condition to be enhanced. The settings of the weighting coefficients relative to different dental conditions may be pre-programmed into a memory of the intraoral scanning system, or, the settings of the weighting coefficients may be controlled or set by a user via a user input of the system.

The composed scan information is ideal for enhancing internal structure such as a dentin-enamel junction, demineralization, cracks, caries lesion, voids, plaque, and restorations. The intraoral scanning system may be configured to determined different composed scan information based on two or more different wavelengths emitted by the projector unit. The one or more processors may be configured to determine a plurality of composed scan information including the composed scan information and a second composed scan information, wherein the composed scan information is different from the second composed scan information. The second composed scan information may be suitable for enhancing a different type of dental condition than the composed scan information, or the second composed scan information may correspond to the same tooth as the composed scan information but captured at a different angle between the dental arch and the image sensor unit.

Each of the plurality of composed scan information may include different enhanced internal structures, and wherein the one or more processors may be configured to label each of the plurality of composed scan information. The label indicates the specific enhanced internal structure of the labelled composed scan information of the plurality of composed scan information. The one or more processors may then be configured to select one or more composed scan information that relates to a specific enhanced internal structure. The one or more processors may receive a user input that corresponds to a specific enhanced internal structure, and the one or more processors may then be configured to display the one or more composed scan information that relates to the specific enhanced internal structure.

The enhanced internal structure may correspond to a dental condition in a patient's teeth of the dental arch, wherein the dental condition corresponds to a crack, demineralization, caries lesion, dentin-enamel junction, voids, plaque, and restorations. For example, composed scan information that includes enhanced caries information would be labelled different than a second composed scan information that includes enhanced restorations information, and soon.

The projector unit may be configured to emit light with different wavelengths during subsequent time periods or during time periods where each time periods is confined to one of the different wavelengths.

The projector unit may include one or more first light sources and at least one or more second light sources, wherein the one or more first light sources is configured to emit light within a first group of time periods at the near-infrared wavelength spectrum, and the one or more second light sources are configured to emit light within a second group of time periods at the visible wavelength spectrum. The handheld intraoral scanning device may be configured to switch between the one or more first light sources and the at least one or more second light sources. The switching may implies turning on and off the light sources.

The light sources may be a combination of one or more of following:
light Emitting Diode (LED),
broadband Light Emitting Diode, and
continuous broadband Light Emitting Diode.

The visible light information may include information that corresponds to reflection from the tooth surface caused by the first visible wavelength and captured by the image sensor unit, or, the visible light information may include information corresponding to fluorescence information emitted by the tooth surface caused by exposure of the second visible wavelength and captured by the image sensor unit.

The projector unit may include at least one or more third light sources, wherein the one or more second light sources may be configured to emit light within a second group of time periods at a first visible wavelength, and the one or more third light sources may be configured to emit light within a third group of time periods at a second visible wavelength. The first visible wavelength may be different from the second visible wavelength, and wherein the visible light information may include information corresponding to reflection caused by the first visible wavelength and captured by the image sensor, and wherein the visible light information may include information corresponding to fluorescence information caused by the second visible wavelength and captured by the image sensor unit.

The one or more processors may be configured to determine the surface information provided by the reflection of the first visible wavelength, and the composed scan information may include a composition of the internal structure information and the visible light information including the reflection of the first visible wavelength, and/or, the composed scan information may include a composition of the internal structure information and the visible light information including the fluorescence information of the second visible wavelength. Thereby, one or more processors may be configured to determine a plurality of composed scan information that includes a summation and/or a difference between the internal structure information, the surface reflection and/or the fluorescence information.

The near-infrared wavelength may be between 701 nm and 2500 nm, the first visible wavelength may be between 380 nm and 700 nm, the second visible wavelength may be between 100 nm and 500 nm, and the visible wavelength may be between 100 nm and 700 nm. In another example, the first visible wavelength may be a white coloured wavelength (430-750 nm), and the second visible wavelength may be a blue-coloured wavelength (i.e. 380 nm-420 nm).

The image sensor unit may include at least one image sensor configured to capture light from within a range of between 100 nm and 2500 nm. The image sensor unit may include a plurality of image sensors, where each of the plurality of image sensors is configured to capture light between 701 nm and 2500 nm, 100 nm and 500 nm, or 380 nm and 700 nm.

A NIR image sensor of the plurality of image sensors may be arranged distantly away from other image sensors of the plurality of image sensors.

The emitted light from the projector unit may be guided via a probe of the handheld intraoral scanning device and through a window in the probe. The NIR image sensor may be arranged in vicinity to the window or above the window and within the probe.

The projector unit may be configured to emit light with different near-infrared wavelengths for the purpose of penetrating the dental arch, i.e. a tooth or teeth, with different depth for the purposed of generating depth information of the enhanced internal structures. For example, it would be of an advantage to know the depth of a caries, a restoration, a crack etc. By determining a plurality of composed scan information, wherein each of the plurality of composed scan information is determined based on internal structure information with different near-infrared wavelengths, the one or more processors may be configured to determine based on the plurality of composed scan information the depth of the enhanced internal structure. For example, each of the composed scan information corresponds to an enhanced internal structure at a depth into a tooth that corresponds to a near-infrared wavelength. The one or more processors is configured to determine the depth based on a depth relation algorithm that includes a relation between depth in a tooth of different type and near-infrared wavelengths. The algorithm receives a near-infrared wavelength that corresponds to a composed scan information and outputs a corresponding depth into the tooth.

The projector unit may be configured to emit light with different near-infrared wavelengths, and the image sensor unit may be configured to capture internal light information from at least the dental arch caused by the different near-infrared wavelengths. The one or more processors may then be configured to determine a plurality of internal structure information, wherein each of the plurality of internal structure information corresponds to reflection caused by a near-infrared wavelength of the different near-infrared wavelengths. The one or more processors may further be configured to determine a plurality of composed scan information, wherein each of the plurality of composed scan information is determined based on each of the plurality of internal structure information and the visible light information.

The one or more processors may be configured to determine depth information of an enhanced internal structure of a tooth or teeth of the dental arch, wherein the determined depth information is based on the plurality of composed scan information.

A maximum depth of an enhanced internal structure may not be determined based on the plurality of composed scan information because the different near-infrared wavelengths do not include the exact wavelength that is able to penetrate the tooth and reach the maximum depth of the enhanced internal structure. In this example, the system may include a memory unit that includes a relation between near-infrared wavelengths and depth, i.e. penetration depth, of different types of teeth, and wherein the one or more processors may be configured to determine a longest near-infrared wavelength of the different near-infrared wavelengths where an enhanced internal structure was identified by the one or more processors in one of the plurality of composed scan information. The one or more processors may be configured to determine an internal depth information of the enhanced internal structure based on the longest near-infrared wavelength and the stored relation between near-infrared wavelengths and penetration depth of teeth. The internal depth information may then be an interpolation of the longest near-infrared wavelength and the stored relation.

The different types of teeth may be incisors, canines, premolars, molars, and third molars.

In another example, the emitted light from the projector unit may be coded structured light that is formed by a grid pattern which the light from the light source(s) is passing through to form coded structured light that is emitted to a dental arch. The one or more processors may be configured to a apply a sequence of coding and decoding algorithm to process the grid pattern. The reflection of the coded structured light may be captured by the image sensor unit, and the internal depth information is determined based on triangulation between point clouds of the coded structured light. The coded structured light may include near-infrared wavelength(s) and/or visible wavelength. The visible light information may include coded structured visible light. The internal light information may include coded structured near-infrared light.

The number of near-infrared wavelengths may be restricted to the size of the handheld intraoral scanning device and the size of the light sources, and thereby, it may be needed to estimate sub internal depth information between the available near-infrared wavelengths of the projector unit or further into a tooth than what the available near-infrared wavelengths allow. Thereby, a trained neural network may be needed to perform such estimation of sub internal depth information. First, the one or more processors is configured to determine a first depth information and a second depth information based on the available near-infrared wavelengths, and then determine a sub internal depth information based on the trained neural network receiving the first and the second depth information. The neural network is configured to determine sub internal depth information of a tooth by interpolation of the received depth information and the knowing of the type of tooth which the received depth information correspond to. The neural network may be trained based on a plurality of composed scan information that are determined based on different near-infrared wavelengths and a relation between near-infrared wavelengths and corresponding penetration depth in different type of teeth of a patient or multiple patients. In another example, the neural network is trained based on X-ray scan information or OCT scan information of a patient or multiple patients. The neural network may include one or more of following topologies, Convolutional Neural Networks (CNN), Three-circle model, persistent homology method and algebraic topology.

For improving the quality of the internal light information, it would an advantage to capture light from different angles between the dental arch and the image sensor unit, and then combine the light from the different angles into a single light information. The combination could be an average or a weighted average of the light from the different angles. The internal structure information may be determined based on multiple captured internal light information, wherein the multiple captured internal light information may be captured by the image sensor unit at different angles between the dental arch and the image sensor unit. The internal structure information may be determined based on an average or a weighted average of the multiple captured internal light information.

The one or more processors may be configured to determine the surface information and/or the fluorescence information based on multiple captured visible light information, wherein the multiple captured visible light information may be captured by the image sensor unit at different angles between the dental arch and the image sensor unit.

The determined surface information and/or the fluorescence information may be based on an average or a weighted average of the multiple captured visible light information.

The emitted light may penetrate a window in the handheld intraoral scanning device, and the system may have a longitudinal axis that is perpendicular to a surface of the window. The emitted light may penetrate the window along the longitudinal axis, and the different angles may be determined between a surface of the dental arch and the longitudinal axis. The longitudinal axis may intersect the surface of the dental arch and the different angles may be formed between the surface and the longitudinal axis.

A composed scan information may normally include an enhanced internal structure that may be displayed in combination with a 2D image (e.g. white light image), a NIR image or a fluorescence image. The displaying may be as an overlay to the 2D image. The enhanced internal structure may be displayed in combination with a 3D model of the dental arch. The displaying may be as an overlay to the 3D model. If a user of the system wants to display different enhanced internal structures on one 2D image or a 3D model, it would be an advantage to determine hybrid scan information that includes a combination of the plurality of composed scan information, wherein at least two of the plurality of composed scan information includes different enhanced internal structures. The hybrid scan information may include a plurality of channels, wherein each of the plurality of channels corresponds to each of the plurality of composed scan information.

When displaying the hybrid scan information, it would be an advantage to colour differently the different enhanced internal structure for making it easier for the user of the system to distinguish between the different enhanced internal structures. Each of the plurality of channels may be assigned by the one or more processors to different colours. For improving the distinguishing of the different enhanced internal structures even more, it would be an advantage that the one or more processors may be configured to weight differently or similarly each of the plurality of channels with a channel weighting coefficient. The channel weighting coefficient determines the contribution of a composed scan information to the hybrid scan information. Furthermore, adjusting the channel weighting coefficient of each of the plurality of channels would result in a change in brightness, contrast and/or transparency of the composed scan information.

The composed scan information may be in the form of a bitmap suitable for being overlayed on a 2D image or a 3D model or a 2D image, such as a 256×256 image, where each pixels may be generated from subtracting and/or addition of two or more different 2D images, i.e. light information captured by different wavelengths. The hybrid scan information may likewise be represented as a bitmap or 2D image, where each pixels may represent multiple channel information.

The plurality of channels may include a first channel that corresponds to a background information, and the background information may include a first composed scan information of the plurality of composed scan information, and a second channel that corresponds to a first stacked information that includes a second composed scan information of the plurality of composed scan information. The first stacked information may be aligned with and stacked on the background information. For example, the enhanced internal structure of the background information may be grayscale coloured information and the first stacked information may be red coloured information that includes a different type of enhanced internal structure than for the background information.

Two or more composed scan information of the plurality of composed scan information may include similar type of enhanced structural information of the same tooth or teeth of the dental arch, and in this example, it would be beneficial to combine the two or more composed scan information into one channel of the plurality of channels of the hybrid scan information. The plurality of channels may include a single composed scan information of the plurality of composed scan information or a combination of multiple composed scan information of the plurality of composed scan information. The combination of multiple composed scan information may for example include a summation of a first and a second composed scan information of the plurality of composed scan information.

The composed scan information may include enhanced internal structural information that is configured to be displayed on a displaying unit of the system. The composed scan information may be processed further by the one or more processors for the purpose of displaying the composed scan information or the plurality of composed scan information. The composed scan information may include further scan information than the enhanced internal structure information. The further scan information may be used for aligning the composed scan information to a background 2D image or a 3D model, or to a second composed scan information for the purpose of being displayed. The one or more processors may be configured to display the composed scan information on a displaying unit of the intraoral scanning system.

The further scan information may include scan information of other parts of a tooth or teeth of the dental arch that does not relate to the enhanced internal structure, and furthermore, the further scan information may include information on gingival, bone and other parts of the dental arch. The further scan information may by structural information of a tooth, gingival or any anatomical structures of a dental arch.

The dental arch may be a curved structure that encompasses the teeth, gingival and bone. The dental arch may be an upper jaw, a lower jaw or both jaws.

The one or more processors may be configured to determine an enhanced internal structure and/or enhanced texture information based on the composed scan information. The enhanced texture information may include enamel, dentin, dentinenamel junction, fetal, root tip, bone, periodontal ligaments, and/or gums.

The one or more processors may be configured to apply enhanced internal structure and/or the enhanced texture information onto the 3D model or a 2D image of a tooth or teeth of the dental arch. The applying of the enhanced internal structure and/or the enhanced texture information onto the 3D model or the 2D image may be provided by identifying one or more reference features in the composed scan information that includes the enhanced internal structure and/or the enhanced texture information, and identifying one or more other reference features on the 3D model or on the 2D image, and aligning the one or more reference features with the one or more other reference features. The identifying and the alignment are provided by the one or more processors.

During a time frame, the projector unit may be configured to emit visible light pulses that include visible wavelength and near-infrared light pulses that include near-infrared wavelength, and between each pulses a dead pulse time may occur. The dead pulse time represent a period where no pulse is being transmitted. During the time frame one or more visible light pulses may be emitted by the projector unit and then after a dead pulse time one or more near-infrared light pulses may be emitted. The captured visible light information and the internal light information that are caused by the emitted light pulses during the time frame do all correspond to the same area of the dental arch. In this example, no alignments of the plurality of composed scan information is needed or a sparsely alignment may be needed. The one or more processor may be configured to provide a warning if the user of the handheld intraoral scanning device is moving the device too fast such that the captured light information within the time frame does not correspond to the same area of the dental arch. During a time frame the visible light information may have a higher resolution than the internal light information during a specific scanning mode, such as a scan mode or a diagnostic mode.

In another example, each of the plurality of composed scan information may include a reference structure that is used for aligning the plurality of composed scan information of the hybrid scan information. The one or more processors may be configured to perform the alignment of the plurality of composed scan information.

The dead pulse time may be below 0.1 ms, below 0.05 ms or below 0.03 ms.

The one or more processors may be configured to determine a 3D model in real time by stitching multiple surface information, and where each of the multiple surface information is determined based on multiple captured visible light information that were captured during the same time frame. A composed scan information may be determined based on internal light information and visible light information that are captured during the same time frame, and thereby, the one or more processors may be configured to display the composed scan information on a 2D image determined based on the visible light information also used for determining the composed scan information.

The one or more processors may be configured to determine an enhanced internal structure and/or enhanced texture information based on the composed scan information. The enhanced internal structure and/or the enhanced texture information may include location, shape and size measure of a dental condition. The one or more processors may be configured to apply the enhanced internal structure and/or the enhanced texture information onto a part of the 3D model. The part of the 3D model includes surface information which has been determined based on visible light information and captured during the same time frame as the visible light information and the internal light information being used for determining the composed scan information.

The one or more processors may be configured to apply the enhanced internal structure and/or the enhanced texture information onto a 2D surface image that includes surface information. The surface information may be determined based on visible light information that has been captured during the same time frame as the visible light information and the internal light information have been captured and used for determining the composed scan information.

The one or more processors may be configured to apply enhanced internal structure and/or the enhanced texture information onto the 3D model or a 2D surface image of a tooth or teeth of the dental arch.

The determined depth information of the corresponding enhanced internal structure and/or the enhanced texture information may be displayed on a displaying unit. For example, a marker may be displayed on the displaying unit, and the one or more processors is configured to move the marker based on a user input via a user input device of the system. When placing the marker over a portion of the enhanced internal structure and/or the enhanced texture information on the 3D model or 2D surface image, the internal depth information may be displayed next to the marker or next to the enhanced internal structure and/or the enhanced texture information. The marker provides an intuitive way of displaying the internal depth information.

The one or more processors may be configured to display on a displaying unit of the system the internal depth information of the corresponding enhanced internal structure and/or the enhanced texture information. The one or more processor may be configured to display on the displaying unit the internal depth information on the 3D model, on the 2D surface image, next to the 3D model, or next to the 2D surface. The different approach on

The marker may be a pointer, a circle with an opening, a cursor, a square with an opening etc.

The one or more processors may be configured to display the three-dimensional (3D) model of the dental arch on a displaying unit of the intraoral scanning system, and a marker over a portion of the 3D model of the dental arch on the displaying unit. The one or more processors may be configured to change a relative position between the marker and the 3D model of the dental arch based on input from a user input device of the intraoral scanning system. The one or more processors may be configured to identify, from both the 3D model of the dental arch and the plurality of composed scan information, wherein each of the plurality of composed scan information includes internal light information and visible light information captured at an angle and position, a composed scan information of the plurality of composed scan information that approximates a relative angle and position between the marker relative to the 3D model of the dental arch. The one or more processors may be configured to display the identified composed scan information determined for the angle and position that approximates the angle and position between the marker relative to the 3D model of the dental arch. In this example, the one or more processors is configured to select the fluorescence information and the internal structure information by comparing the angle and position of the captured visible light information and the internal light information that correspond to the fluorescence information and the internal structure information with the approximated angle and position between the marker and the 3D model. Once again, the marker brings an intuitive way of identifying and displaying relevant composed scan information.

The one or more processors may be configured to display the three-dimensional (3D) model of the dental arch on a displaying unit of the intraoral scanning system, and display a marker (a viewing marker, a cursor) over a portion of the 3D model of the dental arch on a displaying unit of the system. Furthermore, the one or more processors may be configured to continuously, as the user changes the relative position between the marker and the 3D model of the patient's dental arch based on a user input, and identify, from both the 3D model of the dental arch and the plurality of composed scan information, wherein the plurality of composed scan information includes a group of composed scan information determined for internal light information and visible light information taken at the same angle and position relative to the dental arch, a group of composed scan information of the plurality of composed scan information determined at an angle and position that approximate the angle and position of the marker relative to the displayed 3D model of the dental arch. Additionally, the one or more processors may be configured to display on the displaying unit the at least one of the group of composed scan information corresponding to the angle and position that approximate the angle and position of the marker relative to the displayed 3D model of the dental arch. The group of composed scan information may correspond to the same tooth or an area of the dental arch, where each of the composed scan information of the grouped composed scan information may include different enhanced internal structure and/or enhanced textural structure.

The one or more processors may be configured to receive, based on a user input, one or more of the plurality of composed scan information including an internal structure, correlate the one or more of the plurality of composed scan information to the 3D model to associate a location of the one or more of the plurality of composed scan information with a corresponding location in the 3D model, and display the 3D model on a displaying unit of the system showing a section through the 3D model with the one or more of the plurality of composed scan information included on the section, showing internal structures including the internal structure. The one or more of the plurality of composed scan information may be displayed on the 3D model. The section through 3D model may be decided by the location of a marker or of a decisive dental condition determined based on a user input.

The one or more processors may be configured to display, in a user interface of the intraoral scanning system, a label selector that is configured to select one or more of the labelled plurality of composed scan information, based on a user input. The label selector may correspond to a press-release button or a check-box on a touch-screen, a keyboard or any kind of a selection mean that allows the user to select different labels. A label corresponds to a dental condition, such as dental caries, restorations, cracks, dental-enamel junction and other type of internal structures or textural structures. The one or more processors may be configured to select, based on a user input, one or more areas-of-interest on the 3D model, and where each of the area-of-interest corresponds to a location of a tooth or teeth of the dental arch on the 3D model, select one or more composed scan information based on the selected one or more areas-of-interest on the 3D model and the one or more selected labels, and display the one or more composed scan information on a displaying unit of the intraoral scanning system. The user is able to select via the label selector those composed scan information that relates to the selected label, i.e. the selected dental condition that is enhanced in the composed scan information. These selected composed scan information may further relate to one or more area-of-interest on the 3D model, and the one or more area-of-interest may be selected by marking on the 3D model.

The displaying of the composed scan information may include a combination of the composed scan information and the surface information or the fluorescence information. An example could be to display the enhanced internal structure and/or the enhanced texture information of the composed scan information on the surface information or the fluorescence information. The surface information or the fluorescence information could be a 3D model or a 2D surface image. The composed scan information, the surface information or the fluorescence information may correspond to the same tooth or teeth of the dental arch. The surface information or the fluorescence information relating to a surface of the tooth or teeth of the dental arch may be coloured with one or more colours, and the composed scan information may be coloured with one or more colours not relating to the one or more colours of the surface. The one or more processors may be configured to select the one or more colours of the surface and the composed scan information based on a user preference or based on a colour machine learning output of a colour machine learning unit of the system that is trained based on a training data set including user preference of multiple users of the system. The user of the system may be colour blind or has preferences to colour combinations, and in this case, it would be beneficial that the user is able to choose the one or more colours. The colour machine learning may be trained to select optimal colour combinations depending on the shape and type of the enhanced internal structures and/or the enhanced textural information. The colour machine learning unit may be trained by colour preferences of other user's of the system in relation to the shape and type of enhanced internal structure and/or enhanced textural information.

The hybrid scan information may be configured to be displayed on a displaying unit of the intraoral scanning system. The displayed hybrid scan information may include a combination of the hybrid scan information and the surface information or the fluorescence information. An example could be the displaying of the enhanced internal structures and/or the enhanced texture information of the plurality of composed scan information of the hybrid scan information on the surface information or the fluorescence information. The surface information or the fluorescence information could be a 3D model or a 2D surface image. A tooth or teeth of the dental arch may include different type of dental conditions and/or multiple of the same dental conditions, and in this situation, the user may want to display these dental conditions on one 2D image or on a 3D model. The one or more processors may be configured to select, based on a user input, the plurality of the composed scan information of the hybrid scan information to be displayed, and the plurality of the composed scan information may include different type of dental conditions and/or multiple of the same dental conditions, i.e. enhanced internal structures and/or enhanced textural information. The one or more processors may be configured to, based on a user input, add or remove one or more composed scan information to the plurality of composed scan information of the hybrid scan information. The displayed hybrid scan information may include one or more colour representing the surface information or the fluorescence information, and the hybrid scan information may be represented by one or more colours not relating to the one or more colours representing the surface information or the fluorescence information. The one or more processors may be configured to, based on a user input, change the colouring of the one or more colours of the hybrid scan information and the one or more colour of the surface information or the fluorescence information.

The one or more processors may be configured to display the three-dimensional (3D) model of the dental arch on a displaying unit of the intraoral scanning system and to display a marker over a portion of the 3D model of the dental arch on the displaying unit. The one or more processors may be configured to change a relative position between the marker and the 3D model of the dental arch based on input from a user input device of the intraoral scanning system, and to identify, from both the 3D model of the dental arch and the plurality of composed scan information, wherein each of the plurality of composed scan information includes internal light information and visible light information captured at an angle and position, two or more composed scan information that approximates a relative angle and position between the marker relative to the 3D model of the dental arch. Furthermore, the one or more processors may be configured to display the hybrid scan information including the identified two or more composed scan information, wherein the internal light information and the visible light information of the selected two or more composed scan information correspond to the angle and position that approximates the angle and position between the marker relative to the 3D model of the dental arch.

The one or more processors may be configured to display the hybrid scan information including the plurality of composed scan information on a displaying unit of the system, and to display a slider (a viewing marker, a cursor) next to the displayed hybrid scan information, on the displaying unit. Furthermore, the one or more processors may be configured to change a position of the slider based on a user input from a user input device of the intraoral scanning system, and to add or remove a composed scan information to the hybrid scan information based on the position of the slider. The slider provides an intuitive and easy way of add or remove one or more composed scan information to the hybrid scan information based on the position of the slider.

The handheld intraoral scanning device may be configured to emit light with different wavelengths during different time periods. In one time period the near-infrared wavelength may be emitted and in one or more consecutive time periods the emitted light may include visible wavelengths. The different time periods may be confined to a time frame. During the time frame, the projector unit may be configured to emit visible light pulses that include visible wavelength and near-infrared light pulses that include near-infrared wavelength, and between each pulses a dead pulse time may occur. The dead pulse time represent a period where no pulse is being transmitted. During the time frame one or more visible light pulses may be emitted by the projector unit and then after a dead pulse time one or more near-infrared light pulses may be emitted by the projector unit. The captured visible light information and the internal light information that is caused by the emitted light pulses during the time frame do all correspond to the same area of the dental arch. The one or more processor is configured to provide a warning if the user of the handheld intraoral scanning device is moving the device too fast such that the captured light information within the time frame does not correspond to the same area of the dental arch.

The projector unit may include one or more first light sources and at least one or more second light sources, wherein the one or more first light sources may be configured to emit light within a first group of time periods at the near-infrared wavelength, and the one or more second light sources may be configured to emit light within a second group of time periods at the visible wavelength. The first group of time periods and the second group of time periods may be arranged accordingly in the time frame. The time frame may include a first repetition rate of time periods of the first group of time periods and a second repetition rate of time periods of the second group of time periods. The time frame may include a third repetition rate of time periods of the third group of time periods. The third group may correspond to the one or more third light sources of the projector unit. The first repetition rate, the second repetition rate and/or the third repetition rate may be determined by the one or more processors based on a scan mode, wherein the scan mode is determined based on a user input. The scan mode may be one of following modes a first diagnostic mode where the first repetition rate is larger or equal than the second repetition rate, a second diagnostic mode where the first repetition rate and the third repetition rate are larger than or equal the second repetition rate, and a first surface scan mode where the first repetition rate is smaller than the second repetition rate. By adapting the repetition rates to a specific scan mode provides an optimized utilization of the bandwidth of the wireless communication to the scan mode if communicating the captured visible light information and the internal light information directly to an external device, and/or an optimization of the power of the handheld intraoral scanning device as only relevant light for the specific scan mode is being emitted and captured. Furthermore, the adapting of the repetition rates provides an optimized utilization of the processing power of the one or more processors.

The one or more processors may be configured to, based on a user Input, toggle between different scan modes during a scanning session of the dental arch based on a user input. For example, the toggling between the different scan modes during a scanning session may be preferable if the user suddenly decides to perform a diagnose scan of a part of the dental arch during a surface scan session.

The system may include a wireless communication interface between the handheld intraoral scanning device and a processor of the one or more processors or a group of processors of the one or more processors, and wherein the visible light information may be wireless transmitted with a first data rate, and the internal light information may be wireless transmitted with a second data rate. The handheld intraoral scanning device may include a wireless transceiver configured to transmit and/or receive data, power and/or information. The handheld intraoral scanning device may include one processor of the one or more processors, wherein the one processor may be configured to determine the composed scan information, and to wireless transmit the composed scan information to another processor of the one or more processors. The one processor may be configured to generate or updating the three-dimensional model, and wireless transmit the three-dimensional model to the another processor.

The one or more processors may be configured to change the first data rate and/or the second data rate according to a scan mode. The first data rate may be different from the second data rate or the same. By adjusting the data rates of the wireless communication, the one or more processors is configured to optimize the utilization of the bandwidth of the wireless communication to the different scan modes. The visible light information and the internal light information are wireless transmitted with the same data rate or different data rate.

The wireless transmission of the visible light information and the internal light information may be transmitted in groups, wherein a group includes visible light information and corresponding internal light information. Thereby, the one or more processors is configured to align the composed scan information or the hybrid scan information to a specific area of the 3D model or to a specific 2D surface image of the dental arch.

The wireless transmission of the visible light information may include a first identification and the wireless transmission of the internal light information may include a second identification, wherein the composed scan information includes the composition of the internal structure information determined by the internal light information including the second identification and the visible light information including the first identification, and wherein the first identification matches the second identification. Thereby, the one or more processors is configured to determine the composed scan information based on information that relates to the same area of the dental arch. Furthermore, the one or more processor may then align the composed scan information or the hybrid scan information to a specific area of the 3D model or to a specific 2D surface image of the dental arch based on the identifications.

The visible light information and the internal light information may be wireless transmitted asynchronously to a processor or a group of processors of the one or more processors. The asynchronously wireless transmission is relevant when it is not necessary to real time display the composed scan information while real time displaying the 3D model. Thereby, it is possible to maintain a normal moving pace of the handheld intraoral scanning device and to maintain the real time displaying of the 3D model while scanning irrespective of how many different wavelengths are to be emitted by the projector unit. The normal moving pace corresponds to the surface scan mode which includes generating and/or updating a 3D model and no composed scan information is to be generated.

The visible light information and the internal light information may be wireless transmitted synchronously to a processor or a group of processors of the one or more processors. The synchronously wireless transmission is relevant when it is necessary to real time display the composed scan information while real time displaying the 3D model.

The real time displaying implies an immediate display of the 3D model and/or the composed scan information while performing the scanning with the handheld intraoral scanning device. The immediate display includes at most imperceptible delays at the receiving side of the information to be displayed.

The one or more processors may be configured to select one or more modulation schemes for modulating the visible light information and/or the internal light information to be transmitted over a wireless network of the intraoral scanning system. The one or more processors may be configured to select one or more modulation schemes based on a scan mode or information type to be transmitted over the wireless network. Thereby, the one or more processor is able to optimize the modulation of the information to be transmitted wirelessly. To use the optimal modulation will secure the right balance between computational power, bandwidth of the wireless communication and the real time perceptibility of the updating/generating of the 3D model and/or the composed scan information. The one or more modulation schemes may include following modulation schemes:
MOK (M-ary Orthogonal Keying),
ACM (Automatic Coding and Modulation Scheme),
MBOK (M-ary Bi-Orthogonal Keying),
CCK (Complementary Code Keying),
CCSK (Cyclic Code Shift Keying),
PSK (Phase Shift Keying),
PPM (Pulse Position Modulation),
QAM (Quadrature Amplitude Modulation),
OCDM (Orthogonal Chirp Division Multiplexing), and
OFDM (Orthogonal Frequency-Division Multiplexing).

The system may include a user interface configured to receive a user input.

According to the aspects, an intraoral scanning system is disclosed. The intraoral scanning system may be configured to provide composed scan information that includes enhanced enamel demineralization information which may be associated with caries lesions and/or enhanced Dentin-Enamel Junction (DEJ) location information, i.e. enhanced internal structures. The system may include a handheld intraoral scanning device that includes a projector unit configured to emit light with different wavelengths during time periods onto at least a dental arch, wherein the different wavelengths include a near-infrared wavelength, a blue-coloured wavelength and white-coloured wavelengths. Different types of anatomical dental scatters and absorbs the infrared radiation differently. The scattering properties of enamel and dentin in the near-infrared wavelength range enables the possibility to image internal structures of a tooth.

Additionally, dental caries and demineralization scatter transiting NIR light and appear as darker or lighter areas when imaged, depending on the type of detection (transillumination and reflection, respectively). Additionally, when a tooth is exposed to light within the soft UV into blue spectrum, the photons may excite the dental material and induce a fluorescence response, i.e. visible light information that includes fluorescence information. Healthy tooth material will emit a green fluorescence signal when exposed to blue light (e.g., 405 nm) whereas bacteria and their metabolites, may emit a red fluorescence signal, i.e. visible light information that includes fluorescence information. The latter observed for example when assessing caries lesions and old dental plaque where bacteria and their metabolites are present. By filtering the excitation wavelengths from the light exposing the image sensor unit, red and green fluorescence signals is captured.

The projector unit may further include an image sensor unit configured to capture visible and internal light information from at least the dental arch caused by the emitted light of the projector unit. The system may further comprise one or more processors operably connected to the image sensor unit. The one or more processors may be configured to receive the visible light information that relates to the projected white-coloured wavelength and/or the blue-coloured wavelength, and the internal light information relates to the near-infrared wavelength. The one or more processors may be further configured to determine fluorescence information of the dental arch from the visible light information, to determine internal structure information of the dental arch from the internal light information, and to determine composed scan information including a composition of the internal structure information and the fluorescence information.

The visible light information and the internal light information relates to specific wavelengths, and the information may be reflected or emitted (fluorescence information) from an outer surface of a tooth and/or from an inner surface of a tooth, and wherein the inner surface is understood as being inside the tooth, and then captured by the image sensor unit.

It has been shown that by generating composed scan information from fluorescence information and the internal structure information by subtracting and/or adding the information enhances enamel demineralization (caries lesion) information and/or enhanced DEJ information. For example, the composed scan information may include a summation of a first difference between the internal structure information and a red fluorescence information and a second difference between the internal structure information and a green fluorescence information. The composed scan information may be overlayered onto a 3D model or a 2D surface image, and the colour that represents the composed scan information should be in strong contrast to the colours being used for the 3D model and the 2D surface image for further improving the visibility of the enhanced caries and/or enhanced DEJ information. For example, the colour of the composed scan information may be red. Furthermore, the brightness and/or contrast of the first difference and the second difference may be reduced or increased for improving the enhanced effect. The reduction/increase in brightness/contrast may be applied by the one or more processors by weighting coefficient applied to the internal structure information, the green fluorescence information and the red fluorescence information.

The fluorescence information determined based on the visible light information, wherein the visible light information includes blue-coloured wavelength, and the fluorescence information includes green fluorescence information and/or at least red fluorescence information, wherein the green and the red fluorescence information are emitted light from a dental arch that has absorbed the visible light information, i.e. the blue-coloured wavelength.

The composed scan information may include a difference calculation between the internal structure information and the fluorescence information. In this example, the composed scan information result in enhanced enamel demineralization (proximal caries) information. The composed scan information may alternatively include a summation of the internal structure information and the fluorescence information. In this example, the composed scan information includes enhanced DEJ information. In a regular NIR image the DEJ is also clearly seen, however, the thickness of the enamel along an edge of the tooth combined with scattering makes the outer rim of the enamel less transparent, thus creating a ribbon-like structure of transparency along the outer contour of the tooth. This ribbon-like structure is also present in the fluorescent images, especially in the green fluorescence information where the signal is largest. This similarity in ribbon-structure between internal structure information and the fluorescence information is very useful when identifying caries lesions through difference imaging.

The one or more processors may be configured to determine a first difference between the internal structure information and the green fluorescence information and a second difference between the internal structure information and the red fluorescence information, and wherein the composed scan information includes a summation of the first difference and the second difference. In this example, the enhanced enamel demineralization (proximal caries) information in the composed scan information becomes even more enhanced in comparison to the example where the composed scan information includes a difference between the internal structure information and only the red or green fluorescence information. This effect can be understood as follows. The internal structure of a tooth seen from the occlusal surface is partially visible in the near-infrared. The thickness of the enamel looking down along the edge of the tooth from the occlusal surface combined with scattering makes the outer rim of enamel seem less transparent, thus creating a ribbon-like structure of transparency along the outer contour of the tooth. This ribbon-like structure can also be partially present in the fluorescent images, since most of the fluorescent signal comes from the dentin and little signal comes from the enamel. This similarity in ribbon-structure helps to highlight proximal caries lesions when difference imaging between NIR and fluorescence images is applied. The red and green fluorescence signals appear different from different areas of the tooth surface, but the proximal caries lesion shows up in the same way when applying the NIR-Fluo difference. This means that when considering the sum of [NIR-Fluo(R)] and [NIR-Fluo(G)], other structural differences are averaged out (appear less pronounced) while the proximal caries lesion remains pronounced and clear to observe.

The composed scan information may include a summation of the internal structure information, the green fluorescence information and the red fluorescence information, and in this example, the enhanced DEJ information becomes even more enhanced compared to summing only the internal structure information and the one of the fluorescence information, i.e. the green or red fluorescence information. The dentin has the capability to absorb the blue-coloured wavelength and emit green and red fluorescence information, while the enamel is seen as a transparent material that usually does not provide any fluorescence information. For example, a tooth may have a demineralized surface which causes more reflection of for example the blue-coloured wavelength resulting in less light being absorbed by the dentin. This is overcome by the proposed composed scan information.

According to the aspects, an intraoral scanning system is disclosed. The intraoral scanning system may be configured to provide composed scan information that includes enhanced restorations information. The system may include a handheld intraoral scanning device that includes a projector unit configured to emit light at a near-infrared wavelength and white-coloured wavelengths onto at least a dental arch, and an image sensor unit configured to capture visible light information and internal light information from at least the dental arch caused by the emitted light of the projector unit. The system may further include one or more processors operably connected to the image sensor unit. The one or more processors may be configured to receive the visible light information and the internal light information, and to determine, in real time, surface information from the visible light information for generating or updating a three-dimensional (3D) model of the dental arch using the surface information. The one or more processors may be further configured to determine internal structure information of the dental arch from the internal light information, and to determine composed scan information including a composition of the internal structure information and the surface information.

The restoration information may relate to fillings, inlays, onlays, crowns and/or sealants.

The composed scan information includes enhanced restoration information such as, shape, size, location, material, and type. Different materials can have different scattering and transmission properties in different spectral regions, even though they might look similar in one spectral region (e.g., the visible region). One material might be opaque in both visible and NIR regions of the spectrum, while another material might be opaque in the visible and translucent in NIR. It has been shown that the composition including a difference between the internal structure and the surface information enhances one type of restoration (for a filling that could relate to the material it is made of, such as amalgam, gold, metal etc.), and a difference between the surface information and the internal structure enhances another type of restoration (plastic, resin, ceramic etc.). Thereby, by determining a hybrid scan information that includes two composed scan information each relating to different types of restoration, it would then be possible to display different types of restorations on one single 2D surface image or on a 3D model.

The one or more processors may be configured to determine a first type of restorations based on the composed scan information including the difference between the internal structure information and the surface information, and a second type of restorations based on the composed scan information including the difference between the surface information and the internal structure information. It is an advantage to be able to distinguish between different type of restorations as it is an information that is usually filled-out on patient's dental chart, but in this example, the information can automatically be provided on the 3D model or dental chart during or after the scanning session.

The one or more processors may be configured to determine a plurality of composed scan information comprising at least the composed scan information that includes the difference between the internal structure information and the surface information and a second composed scan information including the difference between the surface information and the internal structure information. The plurality of composed scan information may be included in a hybrid scan information.

According to the aspects a method for determining composed scan information is provided. The method comprises;
emitting emit light with different wavelengths during time periods onto at least a dental arch, wherein the different wavelengths include a near-infrared wavelength and a visible wavelength,
capturing visible light information and internal light information from at least the dental arch caused by the visible wavelength and the near-infrared wavelength, respectively,
determining, in real time, surface information from the visible light information for generating or updating a three-dimensional (3D) model of the dental arch using the surface information,
determining internal structure information of the dental arch from the internal light information, and
determining composed scan information including a composition of the internal structure information and the visible light information.

According to the aspects a method for determining composed scan information is provided. The method comprises;
emitting emit light at a near-infrared wavelength, a blue-coloured wavelength and white-coloured wavelengths onto at least a dental arch;
capturing visible light information and internal light information from at least the dental arch caused by the emitted light,
determining, in real time, surface information from the visible light information for generating or updating a three-dimensional (3D) model of the dental arch using the surface information,
determining fluorescence information of the dental arch from the visible light information that relates to the emitted blue-wavelength;
determining internal structure information of the dental arch from the internal light information, and
determining composed scan information including a composition of the internal structure information and the fluorescence information.

According to the aspects a method for determining composed scan information is provided. The method comprises;
emitting emit light with different wavelengths during time periods onto at least a dental arch, wherein the different wavelengths include a near-infrared wavelength and white-coloured wavelengths,
capturing visible light information and internal light information from at least the dental arch caused by the visible wavelength and the near-infrared wavelength, respectively,
determining, in real time, surface information from the visible light information for generating or updating a three-dimensional (3D) model of the dental arch using the surface information,
determining internal structure information of the dental arch from the internal light information, and
determining composed scan information including a composition of the internal structure information and the visible light information.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates an intraoral scanning system;

FIGS. 2A to 2E illustrate different examples of the system;

FIGS. 3A to 3H illustrate different examples of one or more processors;

FIGS. 4A to 4H illustrate different examples of composed scan information images that depict different dental condition;

FIGS. 5A to 5E illustrate different examples of an arrangement of a projector unit;

FIGS. 6A to 6D illustrate different examples of an arrangement including one or more light sources;

FIG. 7 illustrates the arrangement on a tip of a handheld intraoral scanning device;

FIGS. 8A and 8B illustrate different example of an optical arrangement;

FIGS. 9A and 9B illustrate an example of a handheld intraoral scanning device;

FIGS. 10A to 10C illustrate examples on determining depth information;

FIG. 11 illustrates an example of how to improve the quality of a composed scan information;

FIG. 12 illustrates an example of the one or more processors;

FIGS. 13A to 13C illustrates examples of a hybrid scan information;

FIGS. 14A and 14B illustrate examples of hybrid scan information images;

FIGS. 15A to 15C illustrate a graphical interface of a displaying unit;

FIG. 16 illustrates a graphical interface of a displaying unit;

FIG. 17 illustrates a graphical interface of a displaying unit;

FIG. 18 illustrates a graphical interface of a displaying unit;

FIGS. 19A to 19C illustrate a graphical interface of a displaying unit;

FIG. 20 illustrates a graphical interface of a displaying unit;

FIG. 21 illustrates a graphical interface of a displaying unit;

FIGS. 22A to 22D illustrate a wireless communication interface; and

FIGS. 23A and 23B illustrate sequence of emitted light from the projector unit.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intra-oral scan data may be performed by a dental scanning system that may include an intraoral scanning device such as the TRIOS series scanners from 3Shape A/S. The dental scanning system may include a wireless capability as provided by a wireless network unit. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is capable of obtaining surface information by operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled relative to the dentition during a scanning session, such that at least some sets of sub-scans overlap at least partially, in order to enable reconstruction of the digital dental 3D model by stitching overlapping subscans together in real-time and display the progress of the virtual 3D model on a display as a feedback to the user. The result of stitching is the digital 3D representation of a surface larger than that which can be captured by a single sub-scan, i.e. which is larger than the field of view of the 3D scanning device. Stitching, also known as registration and fusion, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental arch and a sequence of images of the different pattern configurations are acquired by one or more cameras located at an angle relative to the projector unit.

Color texture of the dental arch may be acquired by illuminating the object using different monochromatic colors such as individual red, green and blue colors or my illuminating the object using multichromatic light such as white light. A 2D image may be acquired during a flash of white light.

Generally the process of obtaining surface information in real time of a dental arch to be scanned requires the scanning device to illuminate the surface and acquire high number of 2D images. Typically a high speed camera is used with a framerate of 300-2000 2D frames pr second dependent on the technology and 2D image resolution. The high amount of image data needed to be handled by the scanning device to eighter directly forward the raw image data stream to an external processing device or performing some image processing before transmitting the data to an external device or display. This process requires that multiple electronic components inside the scanner is operating with a high workload thus requiring a high demand of current.

The scanning device comprises one or more light projectors configured to generate an illumination pattern to be projected on a three-dimensional dental arch during a scanning session. The light projector(s) preferably comprises a light source, a mask having a spatial pattern, and one or more lenses such as collimation lenses or projection lenses. The light source may be configured to generate light of a single wavelength or a combination of wavelengths (mono- or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental arch. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental arch is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental arch. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental arch in response to the illumination of the dental arch is directed, using optical components of the scanning device, towards the image sensor(s). The image sensor(s) are configured to generate a plurality of images based on the incoming light received from the illuminated dental arch. The image sensor unit may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit or a wired network unit. The wireless network unit is configured to wirelessly connect the dental scanning system to the network comprising the plurality of network elements including the at least one network element configured to receive the processed data. The wired network unit is configured to establish a wired connection between the dental scanning system and the network comprising the plurality of network elements including the at least one network element configured to receive the processed data.

The dental scanning system preferably further comprises a processor configured to generate scan data (such as extra-oral scan data and/or intra-oral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental arch. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental arch. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental arch in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The internal depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance color images, x-ray images, and/or combinations thereof.

The examples illustrated in the below figures may be performed by one or more processors 11.

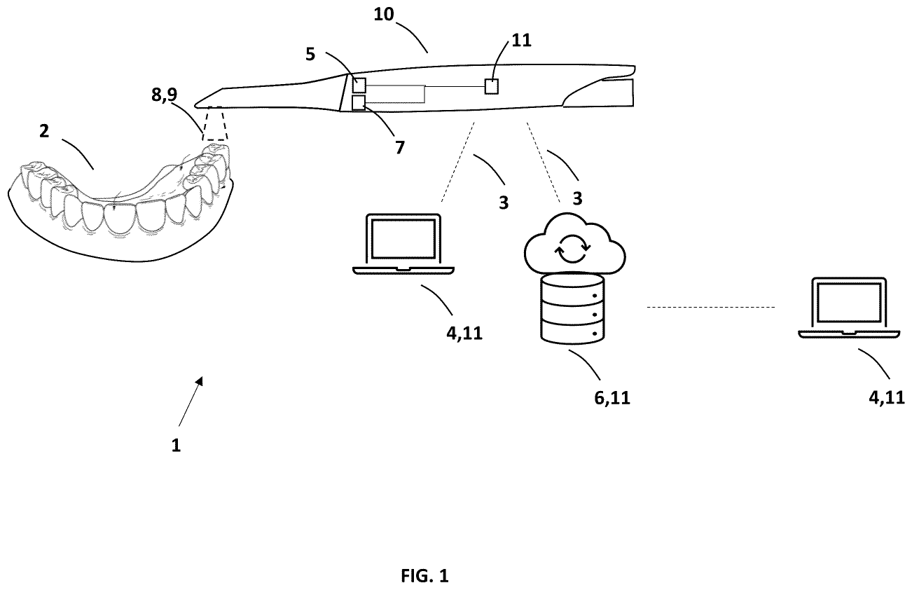

FIG. 1 illustrates an intraoral scanning system 1 that includes a handheld intraoral scanning device 10 configured scan a dental arch 2 of a patient. In this example the scan information generated by the handheld intraoral scanning device 10 is being wired or wireless transmitted 3 to an external computer 4 or to a server 6 which forwards the scan information to an external computer 4. The handheld intraoral scanning device 10 includes a projector unit 5 configured to emit light 8 at a near-infrared wavelength and white-coloured wavelengths onto at least a dental arch. In another, the projector unit 5 is configured to emit light 8 at a near-infrared wavelength and white-coloured wavelengths onto at least a dental arch. In yet another example, the projector unit 5 is configured to emit light 8 with different wavelengths during time periods onto at least a dental arch, wherein the different wavelengths include a near-infrared wavelength and a visible wavelength. The handheld intraoral scanning device 10 may include an image sensor unit 7 configured to capture 9 visible light information and internal light information from at least the dental arch 2 caused by the emitted light 8 of the projector unit 5. In another example, the image sensor unit 7 is configured to capture 9 visible light information and internal light information from at least the dental arch 2 caused by the visible wavelength and the near-infrared wavelength, respectively. The system 10 includes one or more processors arranged in the handheld intraoral scanning device 10, an external computer 4 and/or a server 6 and an external computer 4. The handheld intraoral scanning device 10 may include one processor 11 of the one or more processors, and that processor is configured to process the visible light information and the internal light information into information configured to be transmitted to an external computer 4 or into information configured to be displayed on a displaying unit 4. Furthermore, the external device and the server 6 may each include a processor 11 of the one or more processors.

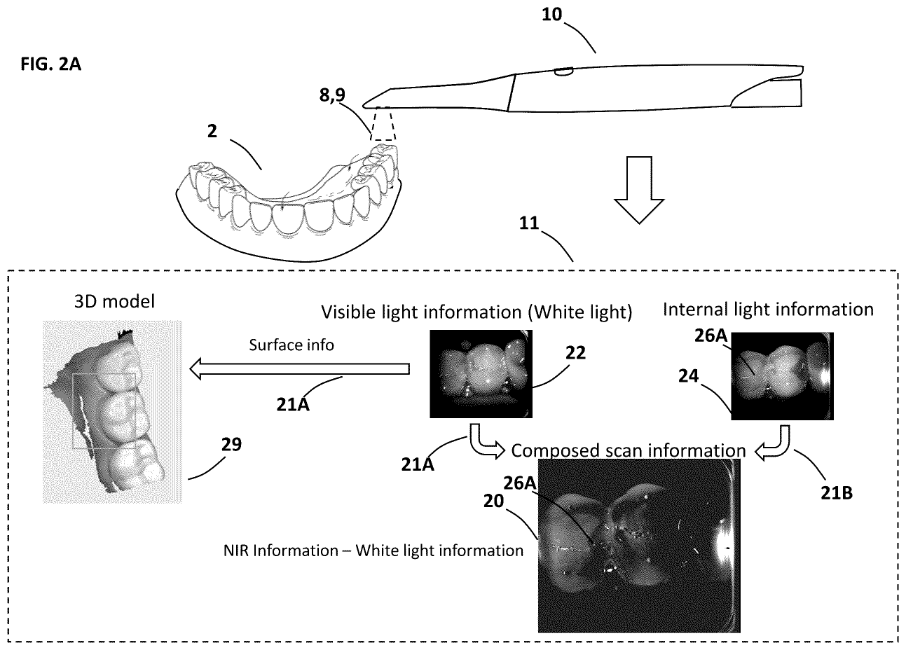

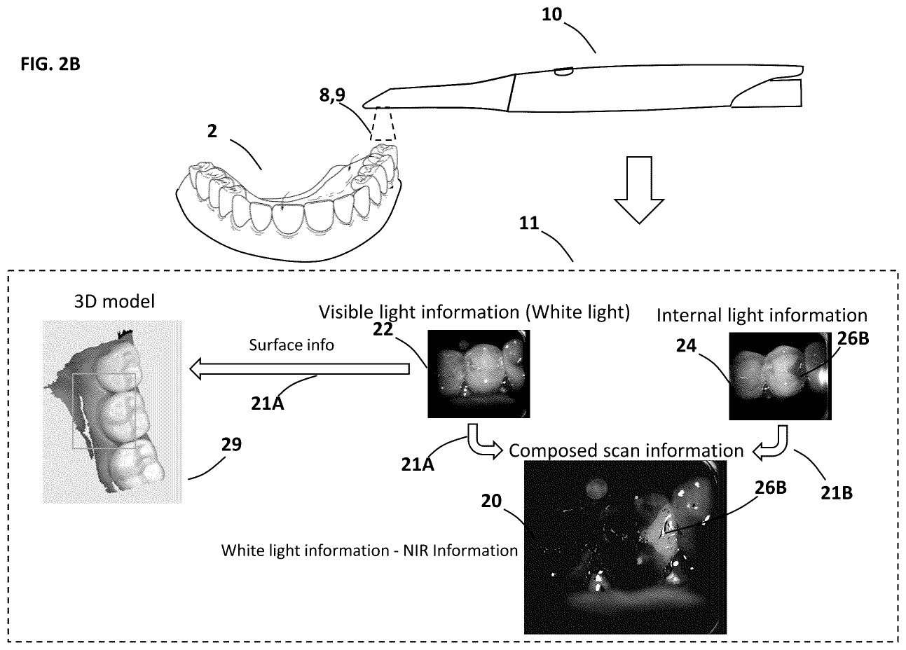

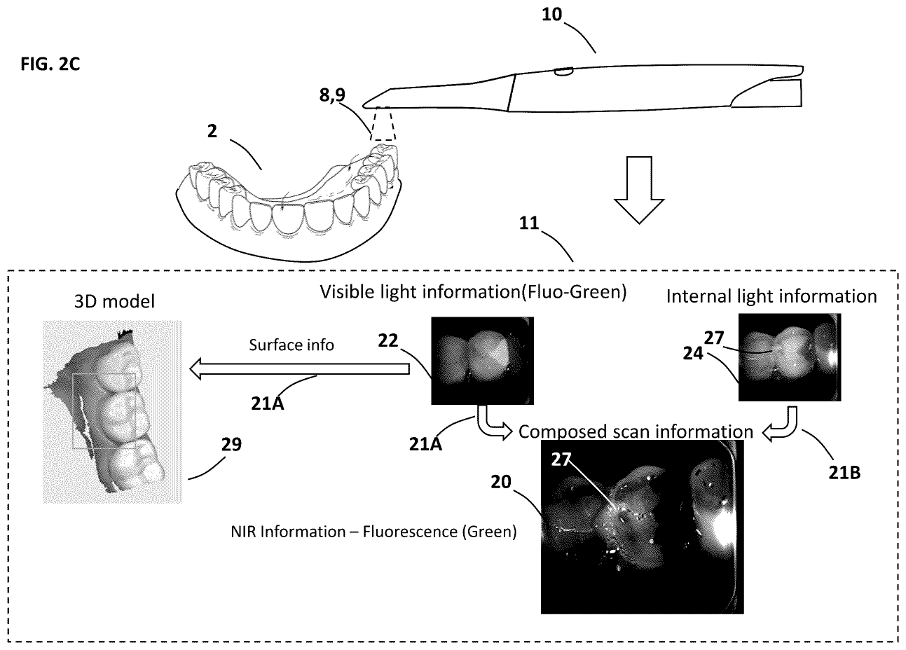

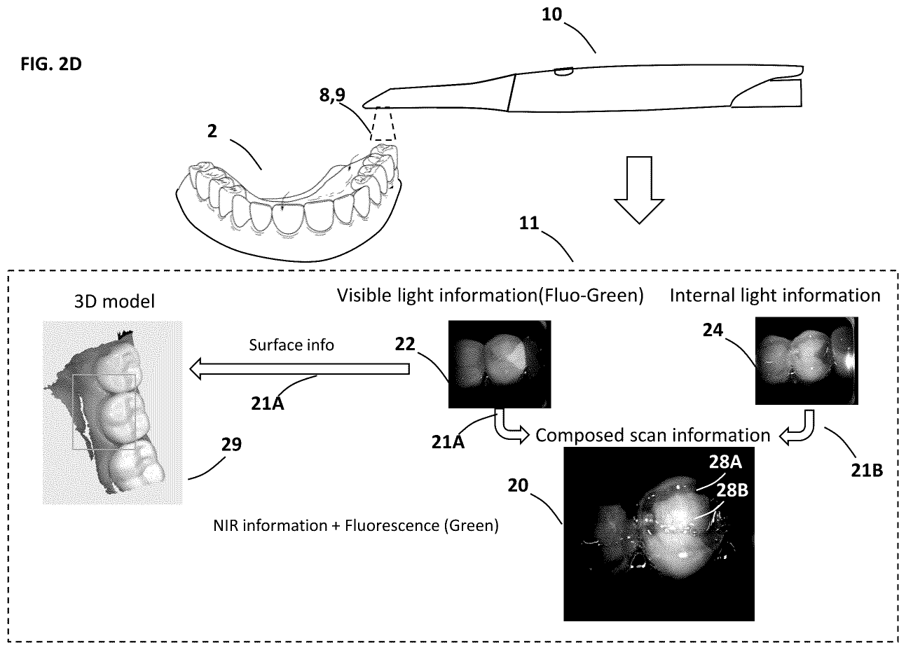

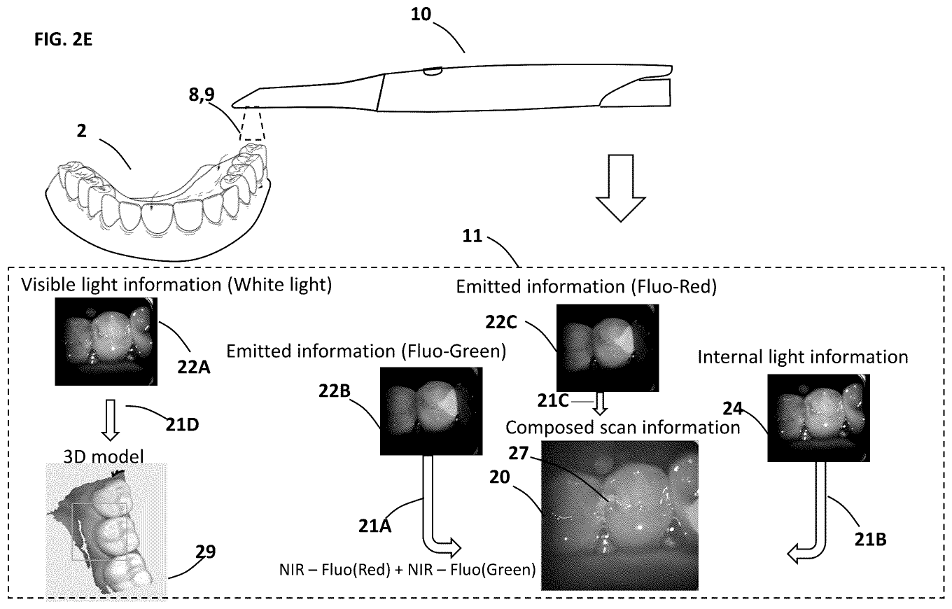

FIGS. 2A to 2E illustrate different examples of the system 1 determining a composed scan information 20. The one or more processors 11 is configured to receive the visible light information 22 and the internal light information 24 from the image sensor unit 7, and determine, in real time, surface information 21A from the visible light information 22 for generating or updating a three-dimensional (3D) model 29 of the dental arch 2 using the surface information. The one or more processors 11 is further configured to determine internal structure information 21B of the dental arch 2 from the internal light information 24, and to determine composed scan information 20 including a composition of the internal structure information 21B and the surface information 21A. In FIG. 2A, the visible light information 22 includes surface reflection, i.e. surface information 21A, provided by white coloured light emitted by the handheld intraoral scanning device 10, and the internal structural formation 21B is provided by reflection of near-infrared light emitted by the handheld intraoral scanning device 10. In FIG. 2A, the composed scan information 20 includes a subtraction of the internal structure information 21B with the surface information 21A. The composed scan information 20 includes enhanced internal structure that is represented by a restoration 26A that is not seen in the near-infrared information 24 but can easily be identified in the composed scan information 20. In FIG. 2B, the composed scan information 20 includes a subtraction of the surface information 21A with the internal structure information 21B. In this example, a different type of restoration 26B is seen in both the internal light information 24 and the composed scan information 20, however, the contrast of the restoration 26B is improved in the composed scan information 20. In FIG. 2C, the projector unit 5 is configured to emit light with near-infrared wavelength and blue-coloured wavelength. In another example, the projector unit 5 is configured to emit light with near-infrared wavelength, blue-coloured wavelength, and white-coloured wavelength. In this present example, the 3D model is generated or updated by surface information 21A provided by fluorescence information 22 that is excited by the emitted light that includes blue-coloured wavelength. The fluorescence information 22 includes green coloured information 21A. The composed scan information 20 includes a subtraction of the internal structural information 21B with the green coloured fluorescence information 21A. The composition 20 is suitable for enhancing the visibility of caries lesion 27. On the near-infrared image 24 the caries lesion 27 can barely be seen, however, on the composed image 20 the visibility of the caries lesion has improved. FIG. 2D illustrates a similar example as FIG. 2C, however, the composed scan information 20 in FIG. 2D is composed differently. In FIG. 2D the composed scan information 20 includes a summation of the green coloured fluorescence information 21A and the internal structure information 21B. In this example the composed scan information 20 includes enhanced textural information about the enamel 28A and dental 28B, and thereby, the Dentin-Enamel-Junction (DEJ). It is easily seen that the enamel 28A and the dental 28B are more clearly seen in the composed scan information image 20 than in the internal light information image 24. In another example, the composed scan information includes a summation of the internal structure information, the green fluorescence information and the red fluorescence information, and the composition, has also shown to improve the visibility of DEJ in relation to a regular or enhanced fluorescence information (, or, in relation to the internal light information 24.

FIG. 2E illustrates an example where the projector unit 5 emits light with blue-coloured wavelength, white-coloured wavelength, and near-infrared wavelength, and the visible light information (22A, 22B, 22C) includes information that relates to surface information 21D corresponding to captured light caused by the white-coloured wavelengths and fluorescence information (21A, 21C) that corresponds to captured light excited by the blue-coloured wavelength. In this example, the fluorescence information (21A, 21C) includes green-coloured fluorescence information 21A and red-coloured fluorescence information 21C. The surface information 21A is used for generating or updating the 3D model 29, and the fluorescence information (21A,21C) are used for determining a composed scan information 20. In this present example, the one or more processors is configured to determine a first difference between the internal structure information 21B and the green fluorescence information 21A and a second difference between the internal structure information 21B and the red fluorescence information 21C, and the composed scan information 20 includes a summation of the first difference and the second difference. The enhance internal structure relates to a caries lesion that has become more visible in comparison to the internal light information image 24 and in the example illustrated in FIG. 2C.

In relation to FIGS. 2A to 2E, the one or more processors 11 is configured to determine fluorescence information (21A,21C) of the dental arch 2 from the visible light information 22.

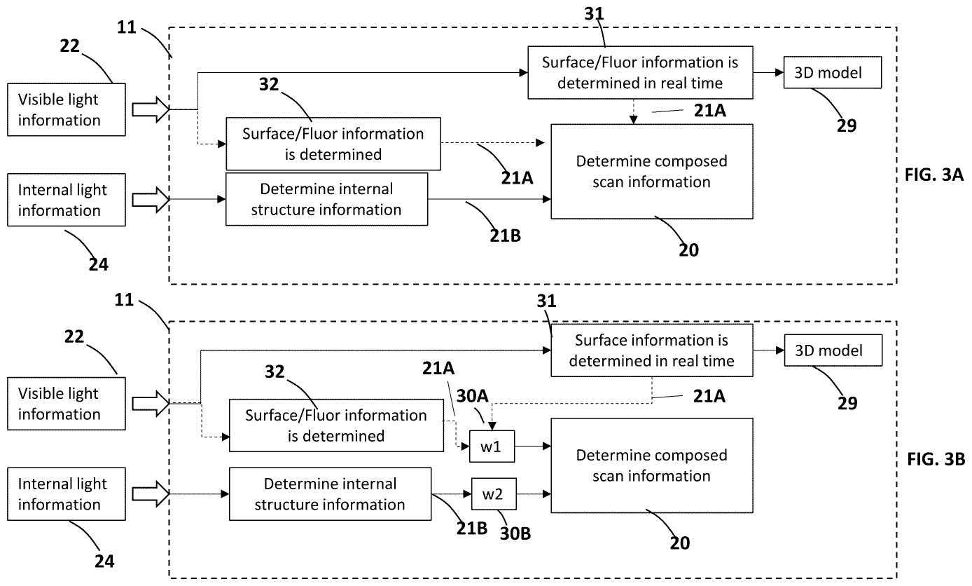

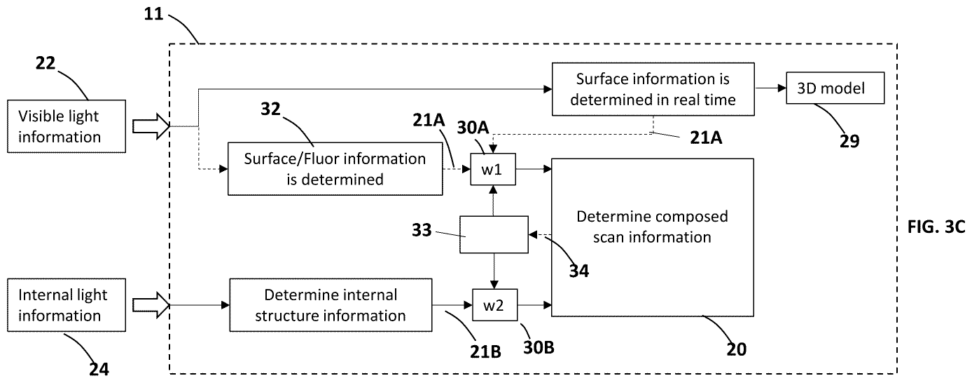

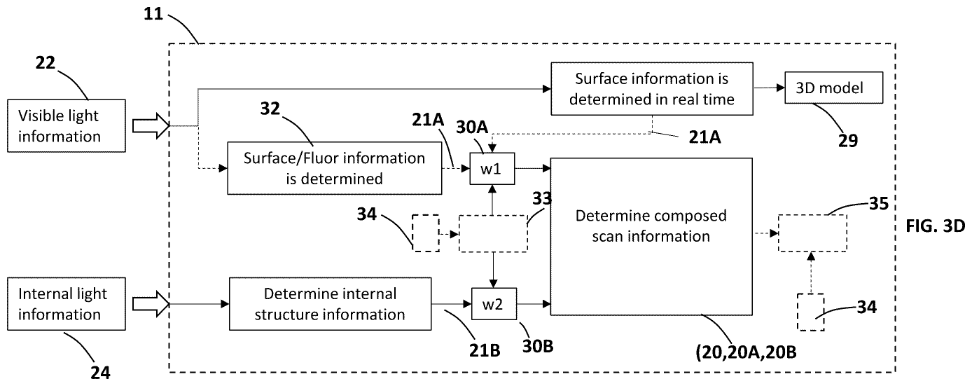

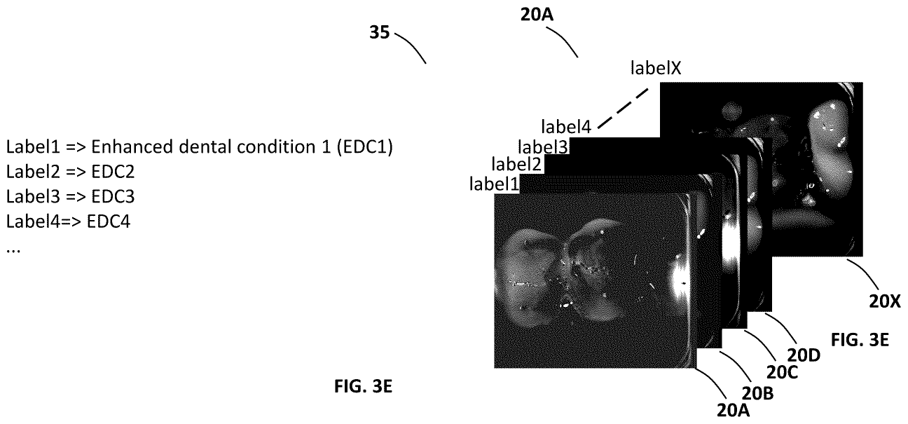

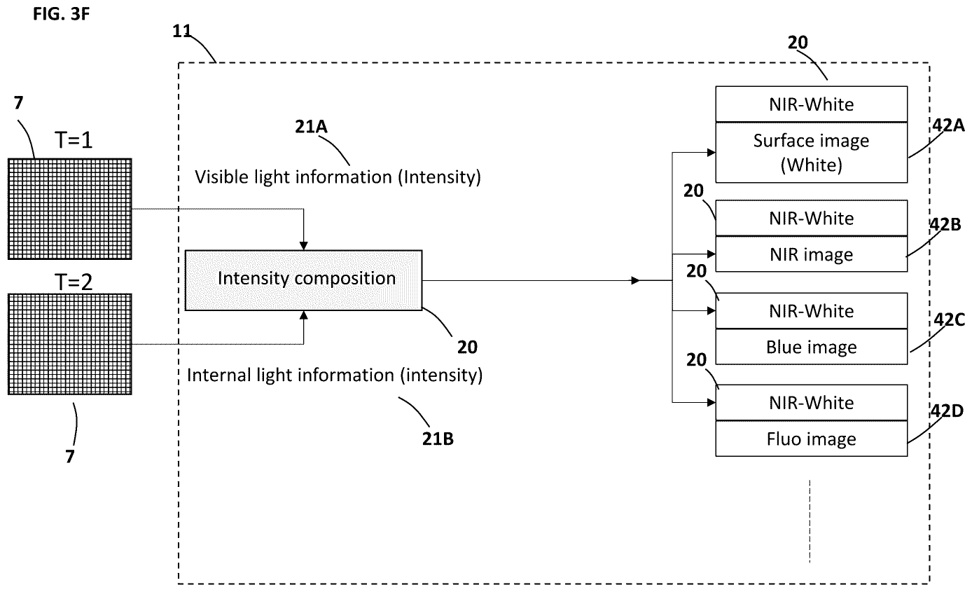

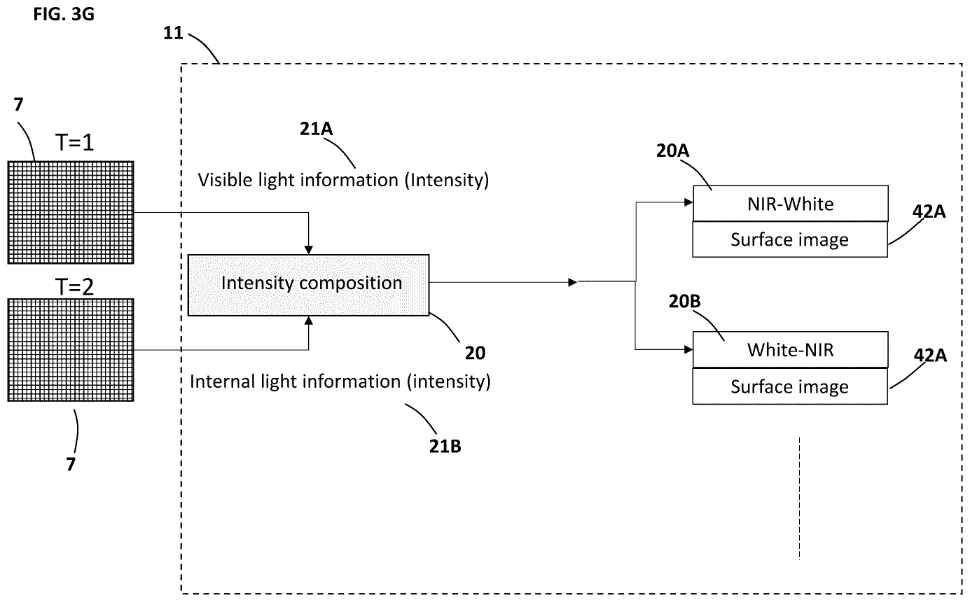

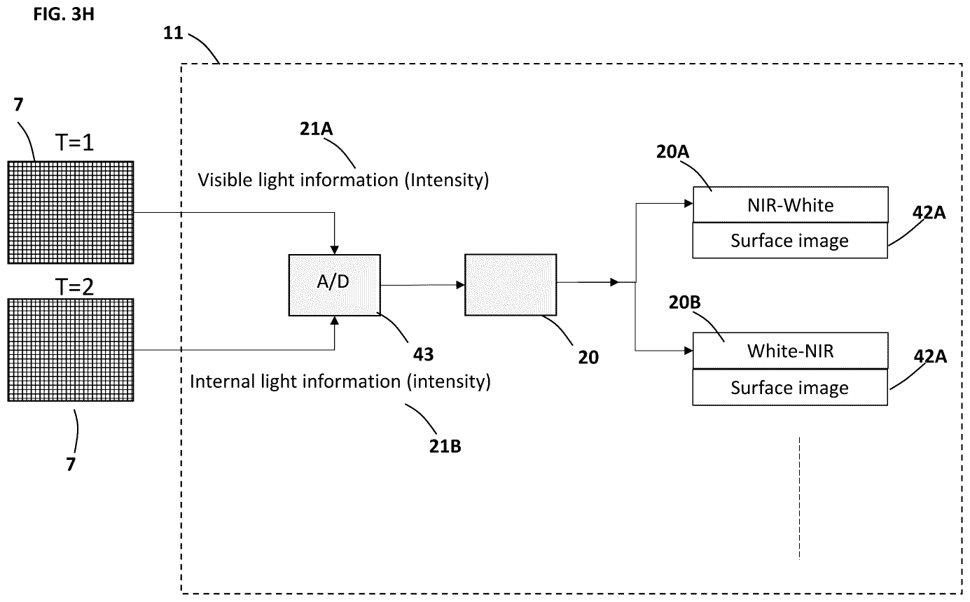

FIGS. 3A to 3D illustrate different examples of the one or more processors 11 receiving the visible light information 22 and the internal light information. The one or more processors 11 is further configured to determine internal structure information 21B based on the internal light information 24, and to determine surface information and/or fluorescence information in real time 31 or not real time 32 based on the visible light information 22. The surface information may be replaced or combined with fluorescence information. The one or more processors 11 is further configured to determine the composed scan information 20 by a composition of the internal structure information 21B and the determined surface information 21A and/or the fluorescence information 21A. The 3D model 29 is either generated or updated by the real time 31 determined surface information and/or the fluorescence information. In FIG. 3B, the determined surface information 21A or the fluorescence information 21A for the composed scan information 20 is weighted by a first weighting coefficient (W1, 30A), and the internal region information is weighted by a second coefficient (W2, 30B). In another example the first weighting coefficient may include a first primary weighting coefficient (W1′) and a first secondary weighting coefficient (W1″). The visible light information may include both surface information and fluorescence information, and in this example, the surface information is weighted by the first primary weighting coefficient (W1′), and the fluorescence information is weighted by the first secondary weighting coefficient (W1″). In FIG. 3C, the one or more processors is configured to change 33 the first and second weighting coefficient (W1, W1′, W1″, W2, 30A, 30B), and wherein the change corresponds to a change in brightness, contrast and/or transparency of the internal structure information and the visible information. The change of the weighting coefficients may be based on a user input. For example, the composed scan information is displayed on a displaying unit of the system 1, and the user is able to change the weighting coefficients via a user interface of the system 1. The user may decide to optimize the weighting coefficients if wanting to improve the visibility of the enhanced internal structures and/or the enhanced texture information on the composed scan information. In another example, the user may decide to optimize the weighting coefficients if wanting to see different type of enhanced internal structures and/or enhanced texture information on the composed scan information. The change of the weighting coefficient may be provided automatically by the one or more processors 11 based 34 on the composed scan information to be determined or based on the internal structure and/or textural information to be enhanced by the one or more processors 11.

In FIG. 3D, the one or more processors is configured to receive different weighting coefficients (W1, W1′, W1″, W2) from a memory 34 of the system 1, and wherein the different weighting coefficients (W1, W1′, W1″, W2) are for different dental conditions to be enhanced in the composed scan information 20. Additionally, or alternatively, the one or more processors 11 is configured to determine a plurality of composed scan information 20 including the composed scan information (20,20A) and a second composed scan information 20B, wherein the composed scan information (20,20A) is different from the second composed scan information 20B. Each of the plurality of composed scan information includes different enhanced internal structures, and wherein the one or more processors 11 is configured to label 35 each of the plurality of composed scan information (20,20A,20B), and the label 35 indicates the enhanced internal structure and/or the enhanced textural information of a composed scan information (20,20A,20B) of the plurality of composed scan information (20,20A,20B).

Th47nhanceced internal structure corresponds to a dental condition in a patient's teeth of the dental arch, wherein the dental condition corresponds to a crack, caries lesion, dentin-enamel junction, voids, plaque, and restorations.

FIG. 3E illustrates an example of the labelling (35, label1, label2, label3, label4) of the plurality of composed scan information (20A,20B,20C,20D,20X), and each labels correspond to different enhanced dental conditions, such as an enhanced internal structure and/or an enhanced textural information.

FIGS. 3F, 3G and 3H illustrate different examples of the one or more processors 11 determining a composed scan information 20 or a plurality of composed scan information (20A,20B). During a first time period T1 the image sensor unit 7 receives for example visible light information 22, and during a second time period T2 the image sensor unit 7 receive internal light information 24, the information are converted into intensity signals (21A,21B) by each of the pixels of the image sensor unit 7 and composed 20 into a composed scan information. In this example, the composed scan information 20 includes a difference between the internal structure information 21B corresponding to near-infrared information and the visible light information 21A including white surface information.

The first composed scan information 20A is configured to be displayed by being layered on top of a white surface image 42A received during the first time period T1. In another example, the composed scan information 20 is layered on top of the internal structural information 42B, on top of blue-coloured excited information 42C received by the image sensor unit 7 during another time period, and/or on top of fluorescence information 42D excited by the blue-coloured wavelength and received by the image sensor unit 7 during another time period. FIG. 3F and FIG. 3G illustrate a similar one or more processors 11, however, in FIG. 3G, the one or more processors 11 is configured to determine a plurality of composed scan information (20A,20B). In this present example, the composed scan information (20A,20B) are layered on top of a surface image 42A which can be either a white surface image of a fluorescence information. Alternatively, the composed scan information (20A,20B) is layered on top of any coloured surface images and/or any coloured fluorescence information such as blue, red or green. FIG. 3H, illustrates that the composed scan information 20 is being determined in digital domain as the intensity signals from the image sensor unit 7 is converted into a digital signal by an Analog-to-Digital converter 43.

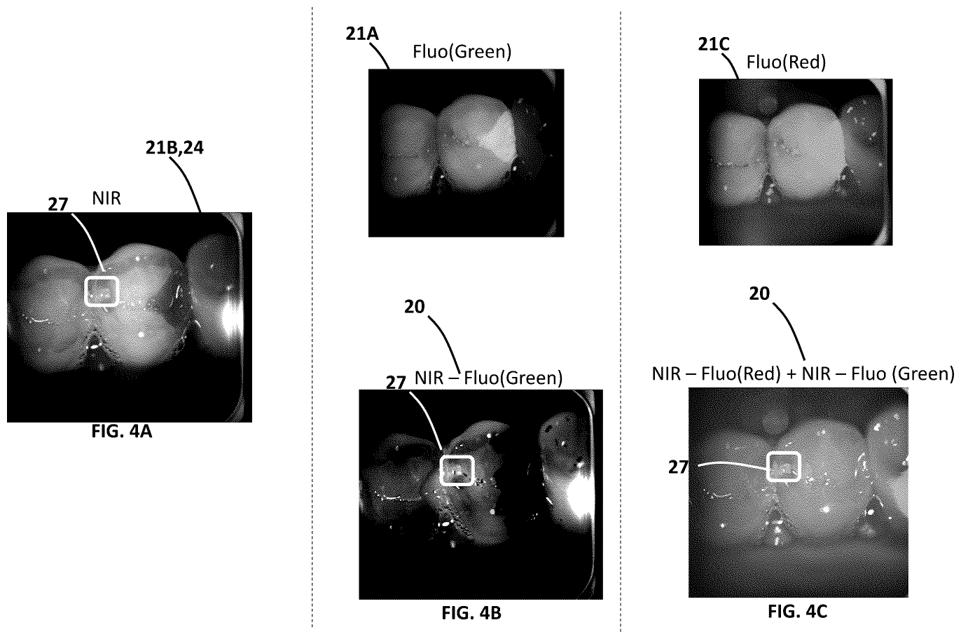

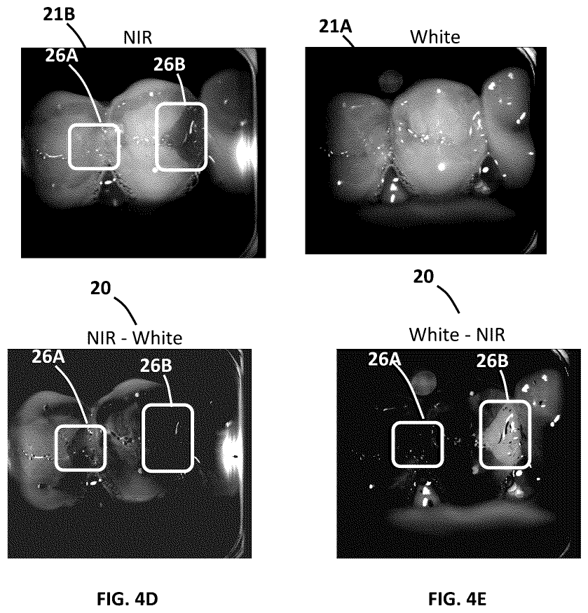

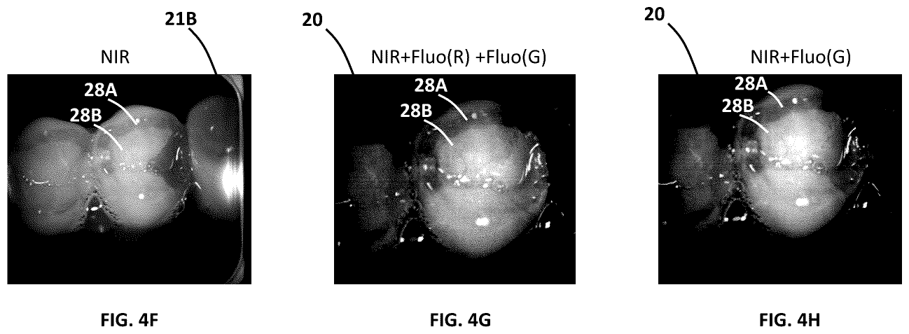

FIGS. 4A to 4H illustrate different examples of composed scan information images that depict different dental conditions. FIGS. 4A, 4B and 4C illustrate different images that depict caries differently. FIG. 4A depicts an internal light information 24 including a near-infrared image of teeth of the dental arch 2, and the image includes information on a caries 27. FIG. 4B illustrates a composed scan information 20 of the same area of the dental arch 2 as illustrated in FIG. 4A, and the composed scan information 20 includes a difference between near-infrared information 21B and green-fluorescence information 21A. The caries is not seen on the green-fluorescence information 21A, and by subtracting the green-fluorescence information 21A from the near-infrared information 21B background information not relating to the caries in the near-infrared information 21B has been reduced, and thereby, the contrast of the caries 27 has improved significantly. FIG. 4C illustrates a composed scan information 20 of the same area of the dental arch 2 as illustrated in FIG. 4B. In this example, the composed scan information 20 includes a first difference between the near-infrared information 21B and a red-fluorescence information 21A, a second difference between the near-infrared information 21B and the green-fluorescence information 21A, and where the first difference is summed together with the second difference. In the composed scan information, the caries has become even more visible than in the two previous examples. In FIG. 4C the composed scan information 20 is applied onto of a coloured surface image of the dental arch 2 captured by the image sensor unit 7 based on emitted light that includes white-coloured wavelength. FIGS. 4D and 4E illustrate different images that depicts restorations differently. FIG. 4D depicts a composed scan information 20 that includes a subtraction of the near-infrared information 21B with a white surface information 21A, and FIG. 4D depicts a composed scan information 20 that includes a subtraction of the white surface information 21A with the near-infrared information 21B. The two teeth depicted on both composed scan information images includes each a restoration, in FIG. 4D the restoration in 26A is seen where in 26B the restoration is gone. In FIG. 4E, the restoration in 26B is seen where in 26A the restoration is gone. In the near-infrared image 21B only the restoration in 26B is seen clearly. By combining the two composed scan information 20 from FIGS. 4D and 4E into a hybrid scan information then it would be possible for the user to identify both restorations (26A, 26B) and not only one restoration 26B. FIGS. 4F, 4G and 4H illustrate different images that depicts DEJ differently. It is clearly seen that the composed scan information 20 in both FIGS. 4G and 4H depicts more clearly the DEJ in comparison to the near-infrared information 21B. The composed scan information 20 in FIG. 4G includes a summation of the internal structure information, i.e. near-infrared information 21B, and green and red fluorescence information (21A, 21C), and in FIG. 4H, the composed scan information 20 includes a summation of the near-infrared information 21B and the green fluorescence information 21A.

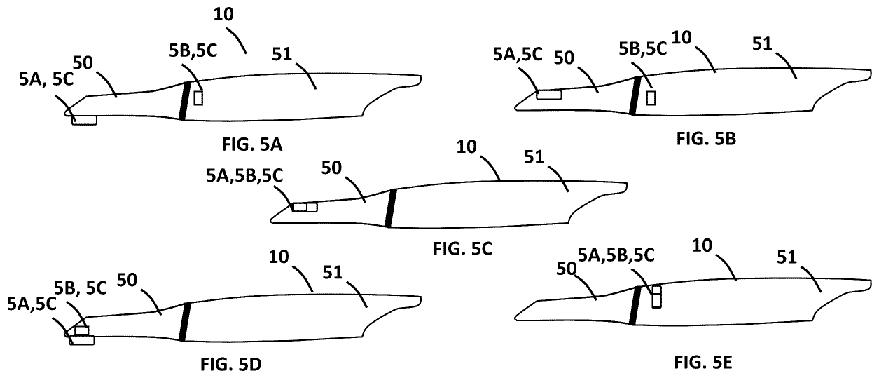

FIGS. 5A to 5E illustrate different examples of the arrangement of the projector unit 5 in or on the handheld intraoral scanning device 10. The handheld intraoral scanning device 10 includes a tip 50 that is either detachably mounted or permanently mounted on a housing 51 of the handheld intraoral scanning device 10. The projector unit 5 is arranged either in the tip 50 or in the housing 51, or in both (50, 51). The projector unit is either in one single unit, as for example illustrated in FIG. 1, but, in other examples, as seen in FIGS. 5A to 5E, the projector unit 5 is arranged in multiple units (5A, 5B). In the examples, illustrated in FIGS. 5A to 5E, the projector unit 5 is divided into two units (5A,5B), but in other examples, the projector unit 5 is divided into more than two units. One example illustrated in FIGS. 5A to 5E, the projector unit includes one or more first light sources 5A and at least one or more second light sources 5B, wherein the one or more first light sources 5A is configured to emit light within a first group of time periods at the near-infrared wavelength, and the one or more second light sources 5B is configured to emit light within a second group of time periods at the visible wavelength, and wherein the visible light information 22 include information corresponding to surface reflection 22A of the dental arch 2 caused by the first visible wavelength and captured by the image sensor unit 7 (not shown). In another example, the visible light information 22 includes information corresponding to fluorescence information (22B,22C) of the dental arch 2 caused by the second visible wavelength and captured by the image sensor unit 7. In another example illustrated in FIGS. 5A to 5E, the projector unit 5 further includes at least one or more third light sources 5C, either arranged together in one single unit 5 together with the other light sources (5A, 5B), or together with either the first light source 5A or the second light source 5B. The one or more second light sources 5B is configured to emit light within a second group of time periods at a first visible wavelength, and the one or more third light sources 5C is configured to emit light within a third group of time periods at a second visible wavelength, and wherein the first visible wavelength is different from the second visible wavelength. The visible light information 22 includes information corresponding to surface reflections 22A of the dental arch 2 caused by the first visible wavelength and captured by the image sensor unit 7, and, the visible light information 22 includes information corresponding to fluorescence information (22B,22C) of the dental arch 2 caused by the second visible wavelength and captured by the image sensor unit 7. Specifically in FIG. 5A, the one or more first light sources 5A and/or the one or more third light sources 5C is arranged at an outer surface of the tip 50 and next to a window of the tip 50. In another example, the one or mor third light sources 5C is arranged within the housing 51. The window is configured to emit and receive light from the projector unit 5 and the dental arch 2, respectively. The image sensor unit 7 (not shown) may be arranged together with the one or more first light sources 5A or within the tip 50 or within the housing 51. The image sensor unit 7 and the one or more first light sources 5A may be arranged in a configuration for performing transillumination of a dental arch 2. The advantage of arranging the images sensor unit 7 and the projector unit 5 on the tip 50, which can be detachably mounted on the housing 51, is that the handheld intraoral scanning device 10 can be adaptable to different scanning purposes and price points of the scanning device 10. For example, a low price point scanning device 10 would be attached to a tip which the main purpose is to protect the optics of the scanning device 10 from being contaminated when inserting the device 10 into the mount of a patient. A high price point scanning device 10 would be attached to a tip as described in FIG. 5A. Thereby, the user is able to shift between a low and a high price point depending on the use of the scanning device 10. In both price points the scanning device 10 is configured to perform scanning for generating or updating 3D model 29. In FIG. 5B, the one or more first light source 5A and/or the one or more third light source 5C is arranged within the tip 50 and not on an outer surface of the tip 50. The advantage of the example depicted in FIG. 5B is similar to FIG. 5A, however, in FIG. 5B, the configuration of the image sensor unit 7 (not shown) and the projector unit (5A,5B,5C) is not suitable for transillumination, but, the hygiene of the tip has improved as the tip 50 is easier to clean. In. FIG. 5C, all three light sources (5A, 5B, 5C) are arranged in the tip, and thereby, the optical path has been reduced between the window of the tip 50 and the light sources (5A, 5B, 5C). Optical elements for guiding the light within the intraoral scanning device 10 becomes simpler, and thereby, the total size of the device 10 is reduced. In this example, the image sensor unit 7 may be arranged in the tip 50 in vicinity to the light sources (5A, 5B, 5C). In FIG. 5D, the one or more first and third light sources are arranged on an outer surface of the tip 50, and the one or more second light sources 5B is arranged within the tip. In this example, the one or more first and third light sources (5A, 5C) are detachably mounted. In FIG. 5E all three light sources (5A, 5B, 5C) are arranged within the housing 51.

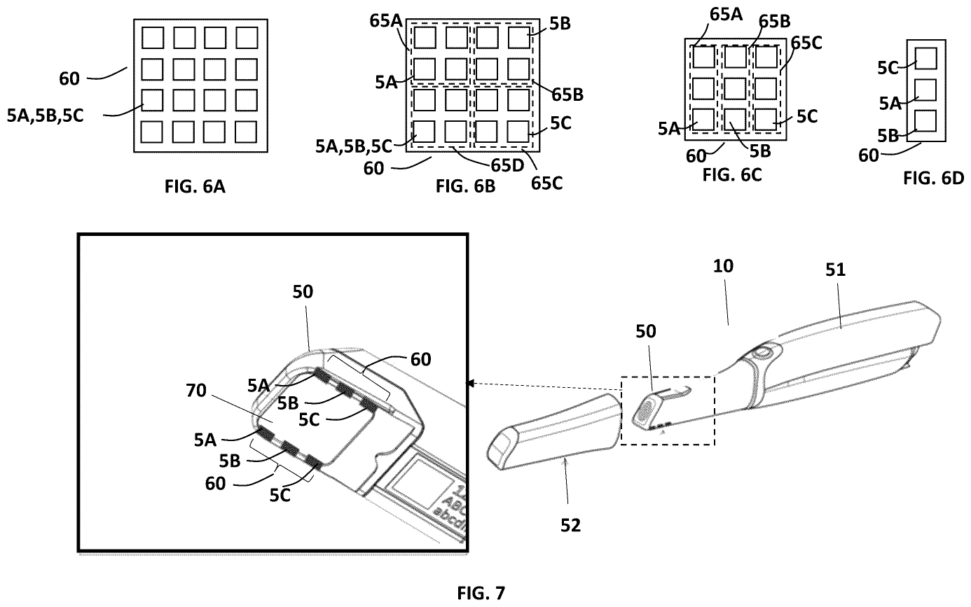

FIGS. 6A-6D illustrate different examples of an arrangement 60 including one or more light sources (5A, 5B, 5C). In FIG. 6A the arrangement 60 includes multiple of light sources that either can be multiple first, second or third light sources (5A, 5B, 5C). In this specific example, the arrangement includes sixteen light sources where each of them is a Light Emitting Diode. In other examples, the arrangement 60 may include three or less, six or less, nine or less, twelve or less, or sixteen or less light sources (5A,5B,5C). In FIG. 6B, the arrangement 60 includes a mix of different light sources that are configured to emit different wavelengths. For example, the arrangement includes a first group 65A of first light sources 5A, a second group 65B of second light sources 5B and a third group 65C of third light sources 5C. In this specific example, the arrangement 60 includes a fourth group 65D of fourth light sources 5D. The fourth group includes different lights sources (5A, 5B, 5C) which may respectively act as a back-up light source for a light source that may break in one of the other groups (65A, 65B, 65C). In FIG. 6C, the arrangement 60 includes nine light sources that are equally divided into three groups (65A, 65B, 65C). Each group represents multiple of light sources that emit the same wavelength. In this example, the first group 65A includes multiple first light sources 5A, the second group 65B includes multiple second light sources 5B, and the third group 65C includes multiple third light sources 5C. In FIG. 6D, the arrangement 60 includes three light sources that are configured to emit different wavelengths.

FIG. 7 illustrates the arrangement 60 on the tip 50 of the scanning device 10 including multiple light sources (5A,5B,5C) distributed on a first side and a second side of a window 70 of the tip 50. In another example, the multiple light sources may be arranged equally around the window 70.

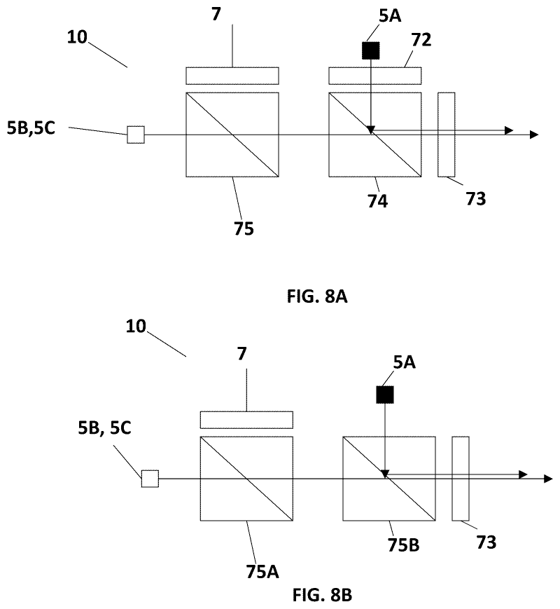

FIGS. 8A and 8B illustrate different example of an optical arrangement within the handheld intraoral scanning device 10. In FIG. 8A, the optical arrangement includes one first light source 5A that is configured to emit light that includes near-infrared wavelength and a second light source (5B, 5C) that is configured to emit light that includes white-coloured wavelengths or blue-coloured wavelength. The light emitted by the second light source (5B, 5C) goes through a first polarizing beam splitter 75, and the emitted light of the first light source 5A goes through a polarizer 72 and received by a beam splitter 74. The emitted light from both light sources (5A,5B,5C) shares the same optical path after the beam splitter 74 and penetrates a quarter wave (QW) plate 73 before reaching the dental arch 2 to be scanned. The near-infrared light is polarized orthogonally to the light coming from the second light source (5B, 5C) before passing the QW plate. The near-infrared light is clockwise circular polarized after the QW plate 73. The polarization state of the QW plate 73 has to change between light coming from the different light sources (5A, 5B, 5C), and thereby, the quarter wave plate may be a rotation quarter wave plate 73 or a liquid crystal based adjustable wave plate. FIG. 8B is similar to FIG. 8A, however, the polarizer 72 and the beamsplitter 74 is combined into a second polarizer beamsplitter 75B. In the examples illustrated in FIGS. 8A and 8B the dental arch 2 is illuminated through a polarizing beam splitter. Due to the polarizing beam splitter, light reflected at the surface of the teeth, so called specular reflected light, cannot reach the image sensor unit 7. This way, only light scattered inside of a tooth which has changed its polarization orientation reaches the image sensor unit 7. Thereby, the quality of the internal light information is improved significantly.

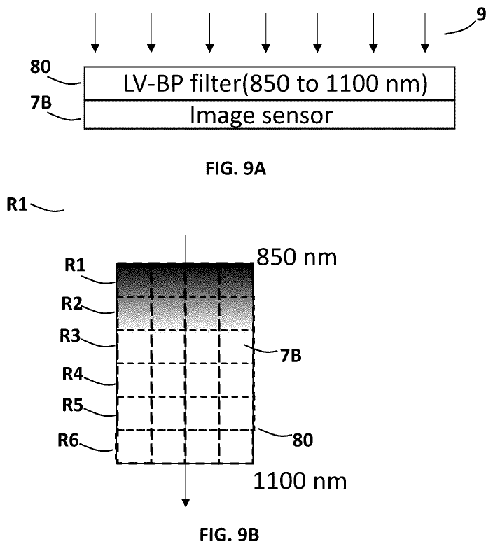

FIGS. 9A and 9B illustrate an example of the handheld intraoral scanning device 10 that includes another image sensor unit 7B, where on-top of the image sensor unit 7B a linear variable bandpass filter 80 is arranged. The another image sensor unit 7B is configured to capture 9 reflected near-infrared wavelengths that are emitted by the projector unit 5. In this example the projector unit includes one or more broadband light emitting diodes that are configured to emit light at wavelengths between 850 nm and 1100 nm. The centre-wavelength of the bandpass filter 80 changes across a longitudinal length of the filter 80 and not along a horizontal length of the filter 80. The filter 80 is aligned with the another image sensor unit 7B such that each row of pixels (R1-R6) are confined to a specific centre-wavelength of the BP filter 80. For example, the pixels in row R1 are confined to a centre-wavelength of around 850 nm, and the pixels in row R6 are confined to a centre-wavelength of around 1100 nm. With a single emitted light pulse from the broadband light emitting diode the another image sensor unit 7B is configured to capture multiple reflections that include wavelengths between 850 nm and 1100 nm, i.e. internal light information 24, and thereby, the one or more processors 11 is configured to determine depth information of an enhanced internal structure of a tooth or teeth of the dental arch 2 based on the captured multiple internal light information 24. First, the one or more processors 11 is configured to determine a plurality of internal structure information based on the captured internal light information caused by the different near-infrared wavelengths captured by the image sensor unit 7B. Secondly, the one or more processors 11 is configured to determine a plurality of composed scan information (20A,20B), wherein each of the plurality of composed scan information (20A,20B) is determined based on each of the plurality of internal structure information and the visible light information. The plurality of composed scan information (2A,2B) corresponds to different near-infrared wavelengths, and each of the near-infrared wavelengths corresponds to a penetration depth in a tooth. The relation between near-infrared wavelengths and the corresponding penetration depth in a tooth are stored in a memory unit of the system 1. The one or more processors 11 is configured to identified in one or more of the plurality of composed scan information (2A,2B) an enhanced internal information. The one or more processor is then able to determine the longest near-infrared wavelength which an enhanced internal structure could be identified in one of the plurality of composed scan information, and then, to determine an internal depth information based on the longest near-infrared wavelength and the stored relation between near-infrared wavelengths and the penetration depth in a tooth of each of the near-infrared wavelengths.

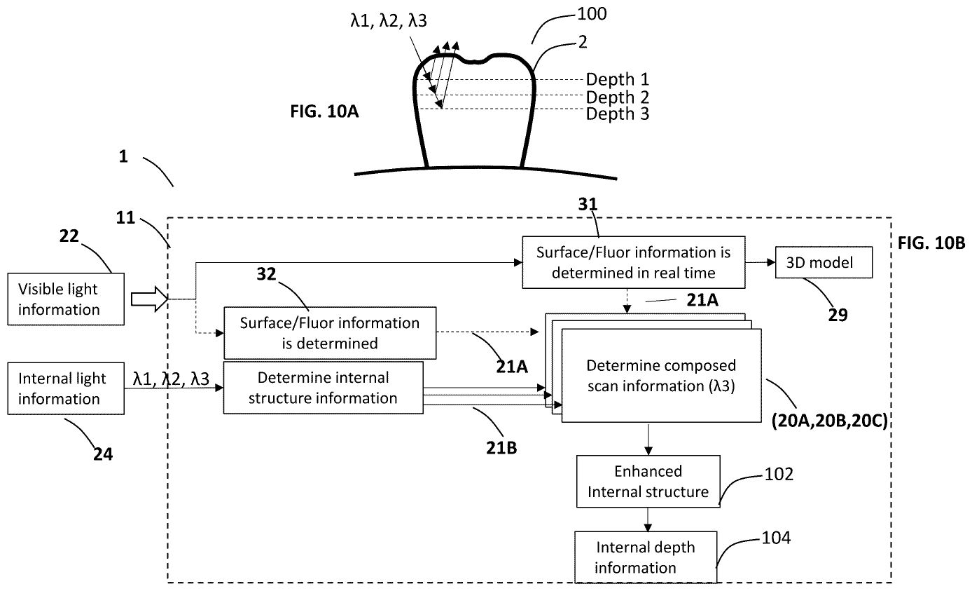

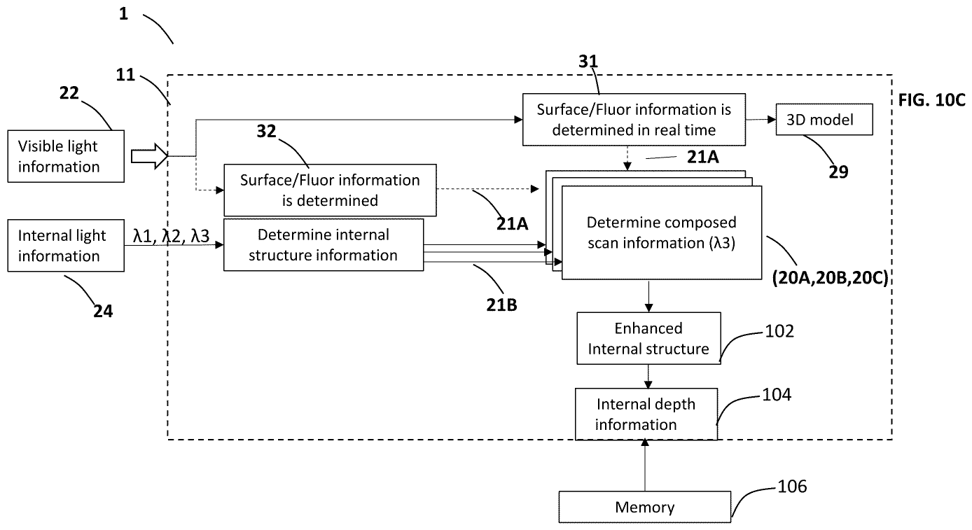

FIG. 10A illustrates the relation 100 between near-infrared wavelengths (λ1, λ2, λ3) and corresponding penetration depths (Depth1, Depth2, Depth3) in a tooth. The relation is stored in a memory unit of the system 1. FIG. 10B illustrates the one or more processors 11 configured to determine depth information 104 of an enhanced internal structure 102 identified in the plurality of composed scan information (20A,20B,20C). In this specific example, the one or more processors 11 receives at least three internal light information captured based on three different near-infrared wavelengths (λ1, λ2, λ3) and visible light information 24 captured based on white-coloured wavelengths or blue-coloured wavelength and based on the at least three internal light information 24 at least three internal structure information 21B are determined. Based on the at least three internal structure information 21B and the visible light information 21A a plurality of composed scan information (20A, 20B, 20C) is determined. The one or more processors 11 is further configured to identify an enhanced internal structure 102 in one or more of the plurality of composed scan information (20A, 20B, 20C), and to determine 104 a longest near-infrared wavelength of the different near-infrared wavelengths (λ1, λ2, λ3) where the enhanced internal structure is identified in one of the plurality of composed scan information (20A,20B,20C). Based on the longest near-infrared wavelength, the one or more processors 11 is configured to determine the internal depth information of the enhanced internal structure. In FIG. 10C the one or more processors 11 is configured to determine 104 the depth internal information of the enhanced internal structure based on the longest near-infrared wavelength and a relation between near-infrared wavelengths and corresponding penetration depth in teeth stored in a memory unit 106 of the system 1. In this specific example, the system 1 is limited to three different near-infrared wavelengths. In another example, the number of different near-infrared wavelengths in the system 1 is limited by the resolution of the image sensor unit 7B and the resolution of the linear variable bandpass filter. It may then be needed to estimate sub internal depth information of an enhanced internal structure between or longer than the available near-infrared wavelengths (λ1, λ2, λ3) of the projector unit 5. Thereby, a trained neural network may be needed to perform such estimation. The trained neural network is part of the system 1. First, the one or more processors 11 is configured to determine a first depth information and a second depth information 104 based on the composed scan information 20A and a second composed scan information 20B of the plurality of scan information (20A,20B,20C), respectively, and then determine a sub internal depth information 104 based on the trained neural network receiving the first and the second depth information. The neural network is trained based on a plurality of composed scan information that are determined based on different near-infrared wavelengths and a relation between near-infrared wavelengths and corresponding penetration depth in teeth of a patient or multiple patients. In another example, the neural network is trained based on X-ray scan information or OCT scan information of a patient or multiple patients. The neural network may include one or more of following topologies, Convolutional Neural Networks (CNN), Three-circle model, persistent homology method and algebraic topology.

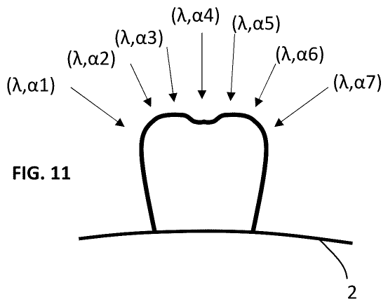

FIG. 11 illustrates an example on how to improve the information of the composed scan information 20. In this example, the captured internal light information 24 has been accomplished at different angles (α1, α2, α3, α4, α5, α6, α7) between the dental arch 2 and the image sensor unit 7 (not shown). The one or more processors 11 is then configured to combine the captured internal light information 24 from the different angles. The combination could be an average or a weighted average of the captured internal light information 24 from the different angles.

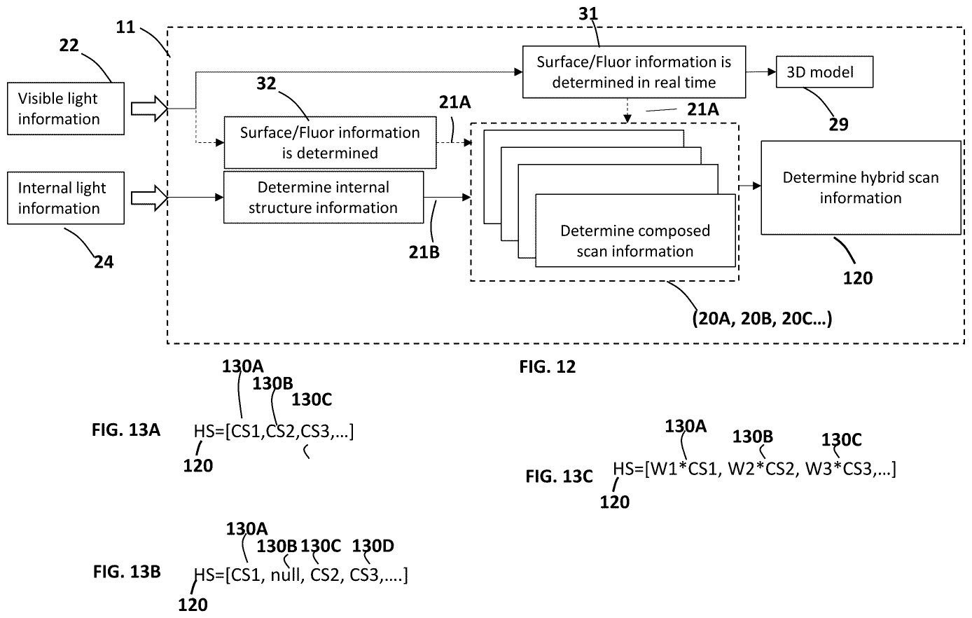

FIG. 12 illustrates the one or more processors 11 that is configured to determine hybrid scan information 120 of the plurality of composed scan information (20A,20B,20C). The hybrid scan information 120 includes a plurality of channels in which each of the plurality of composed scan information is assigned to. The result is an image including the hybrid scan information 120 wherein the internal structure information and/or enhanced textural information are layered on top of each other. Each of the plurality of channels is assigned to a colour. The colour may be different. FIGS. 13A to 13C illustrate the plurality of channels (130A-130C) of the hybrid scan information 120. In FIG. 13A, the hybrid scan information 120 includes at least three-layered composed scan information (CS,20) assigned to at least three channels (130A,130B,130C) of the plurality of channels (130). In FIG. 13B, the hybrid scan information 120 includes again at least three composed scan information assigned to at least three channels (130A,130B,130C,130D), however, in this example, channel 2 (130B) is empty as the colour assigned to channel 2 is not ideal for visualizing the enhanced internal features in the three composed scan information. In FIG. 13C, each of the plurality of channels (130A130B130C) are weighted by a channel weighting coefficient (W1,W2, W3). The one or more processors 11 is configured to change the weighting coefficients (W1, W2, W3) such that the brightness, contrast and/or transparency are changed of the plurality of composed scan information.

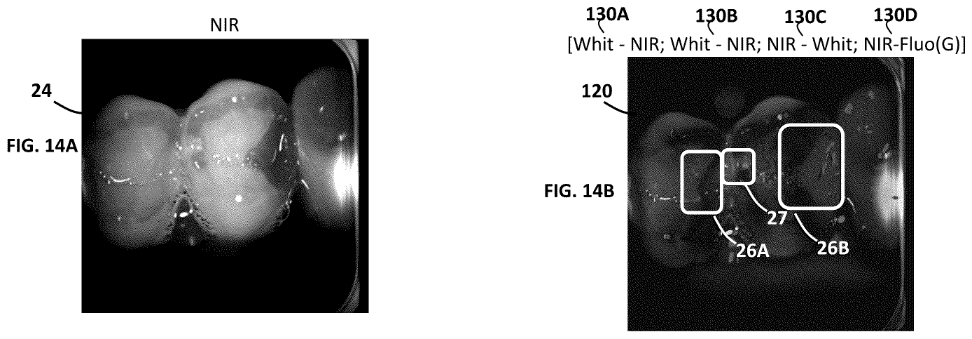

FIG. 14A depicts a near-infrared image that includes internal structure information 24, and FIG. 14B depicts a hybrid scan information 120 that includes four composed scan information that are assigned to different channels (130A,130B,130C,130D). In this example, the four composed scan information are following:
Channel 1=>is assigned to greyscale and includes a first composed scan information 20A that comprises a difference between a white-coloured surface information 21A and internal structure information 21B;
Channel 2=>is assigned to blue and includes a second composed scan information 20B that comprises a difference between a white-coloured surface information 21A and internal structure information 21B;
Channel 3=>is assigned to blue and includes a third composed scan information 20C that comprises a difference between internal structure information 21B and white-coloured surface information 21A; and
Channel 4=>is assigned to blue and includes a fourth composed scan information 20D that comprises a difference between internal structure information 21B and green-coloured fluorescence information 21A.

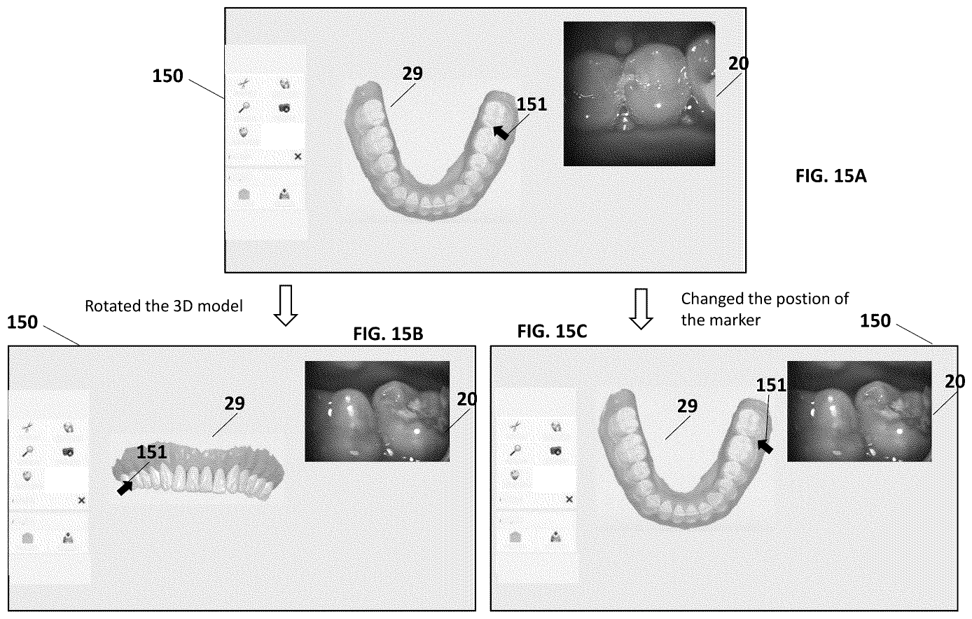

FIGS. 15A, 15B and 15C illustrate a displaying unit 150 that is configured to display, on a displaying unit 150 of the system 1, a composed scan information 20 based on a position of a marker 151 and a relative angle between the marker 151 and the 3D model 29. The one or more processors 11 is configured to change the relative angle by either rotating the 3D model, see FIG. 15B or changing the position of the marker, see FIG. 15C. Furthermore, the one or more processor is configured to identify, from both the 3D model 29 of the dental arch 2 and the plurality of composed scan information (not shown), wherein each of the plurality of composed scan information includes internal light information and visible light information captured at an angle and position, a composed scan information 20 of the plurality of composed scan information that approximates a relative angle and position between the marker 151 relative to the 3D model 29 of the dental arch 2; and display the identified composed scan information 20 determined for the angle and position that approximates the angle and position between the marker 151 relative to the 3D model 29 of the dental arch 2.

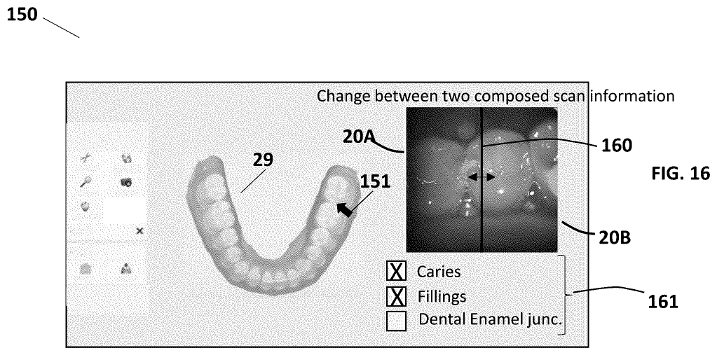

In FIG. 16, the displaying unit 150 is displaying a slider 160, a label selector 161 and a marker 151. In this specific example, the label selector 161 is configured to select one or more composed scan information that relates to one or more enhanced internal structures, such as caries, restorations and/or DEJ. Based on a position of the marker and the selection made by the label selector 161, the one or more processors 11 is configured to select one or more composed scan information (20A, 20B, 20C) to be displayed on the displaying unit 150 that relates to the relative position between the marker and the 3D model and to the selected enhanced internal structures. In this example, the user of the system 1 has specifically selected via the label selector 161 that the displayed composed scan information should include enhanced internal structure information that relates to caries and restorations, and by moving the slider 160, the user is able change between the two composed scan information (20A,20B) to be displayed. In this example, the slider 160 is arranged on top of the composed scan information (20A, 20B), but in other examples, the slider 160 could be arranged next to the composed scan information (20A,20B). The slider 160 brings an intuitive way of changing between different composed scan information to be displayed.

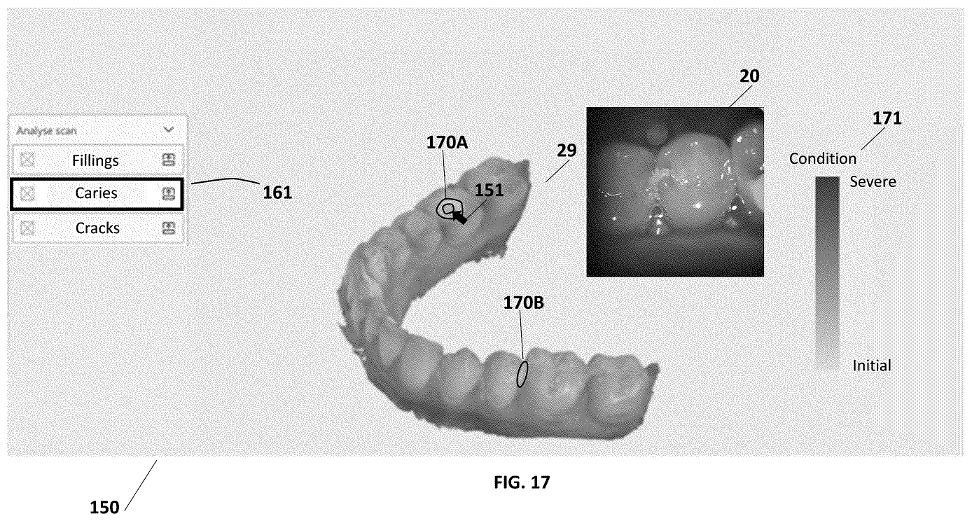

In FIG. 17 the one or more processors 11 has identified enhanced internal structure information of a plurality of composed scan information (20A,20B,20C) and aligned the enhanced internal structure information (170A, 170B) on a 3D model 29 visualized on the displaying unit 150. The type of enhanced internal structure information has been selected by the label selector 161. The condition of the displayed enhanced internal structure information (170A,170B) is indicated by a colour scale 171 on the 3D model 29. In this example, the user wants to display 150 caries lesions on the 3D mode 29 by selecting caries on the label selector 161. The 3D model depicts that the patient has one severe caries lesion 170A and an initial caries lesion 170B. In another example, the user is able to select 151 on the 3D model an enhanced internal structure which should be displayed on a 2D image including the corresponding composed scan information 20. The one or more composed scan information 20 may be displayed next to the 3D model or on the 3D model.

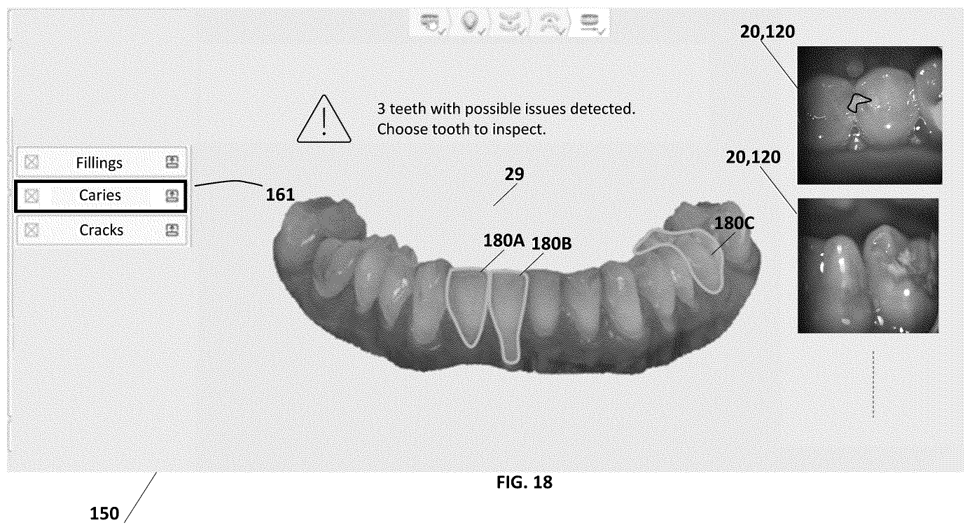

In FIG. 18 the enhanced internal structure information (170A,170B) are not directly visualized on the 3D model 29. Instead, a tooth that includes at least one enhanced internal structure information (170A,170B) identified by the one or more processors 11 based on one or more composed scan information 20 is highlighted on the 3D model. One example of highlighting a tooth is by applying a colour to the tooth, a circumferential marking line around the tooth, a visual marker on the tooth, such as a star, an exclamation marker etc. By clicking on the highlighted tooth one or more images including one or more composed scan information 20 or a hybrid scan information 120 that relates to the highlighted tooth is displayed next to the 3D model 29. The highlighting of the teeth may be depended on a selected dental condition via the label selector 161. In this example, the user wants to highlight teeth that has a caries, and the user has clicked on one of the highlighted tooth and two images including composed scan information of the highlighted tooth is seen from two different angles. In another example, the user may have selected two dental conditions via the label selector 161, and in this example it would be suitable to display a hybrid scan information 120 that includes enhanced internal structures that relate to the selected dental condition. And/or, two or more composed scan information (20A,20B) that relate to the selected dental condition may be displayed next to the 3D model.

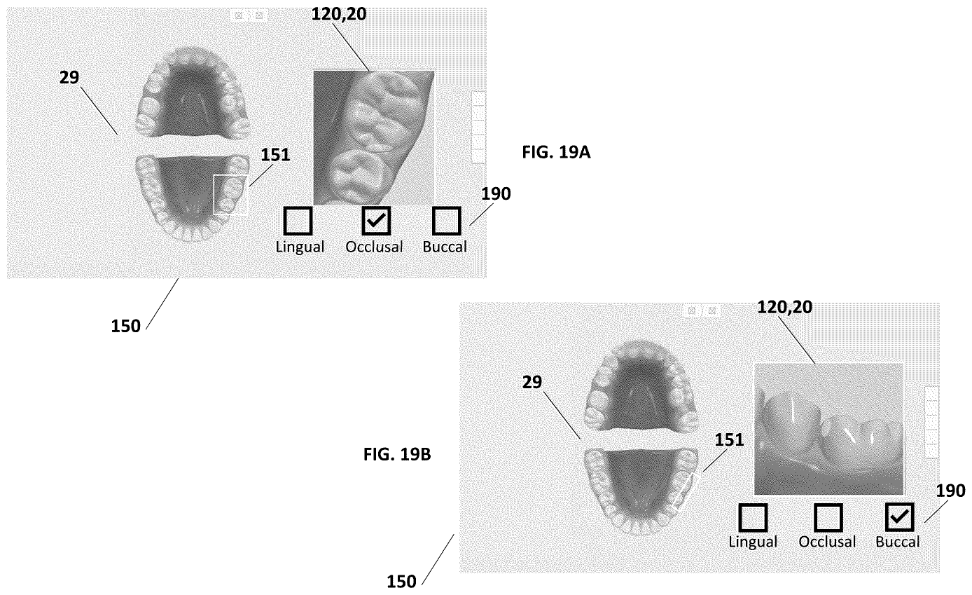

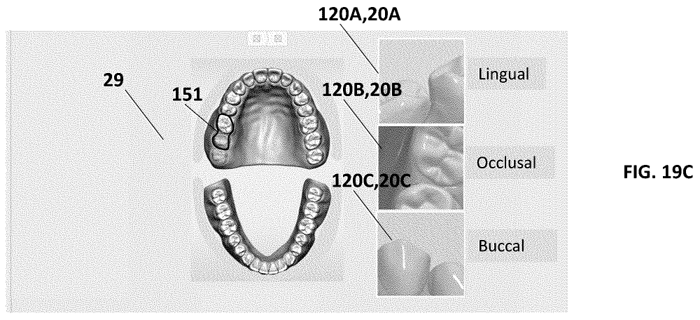

FIGS. 19A and 19B illustrate different examples of the displaying unit 150 that displays the 3D model 29 including both the lower and upper jaw, and next to the 3D model 29 a composed scan information 20 or a hybrid scan information 120 is displayed according to a position of the marker 150 and a selected viewing angle via a viewing angle selector 190 of the displaying unit 150. In FIG. 19A the use has selected one area on the 3D model 29 with the marker 151 and an occlusal viewing angle 190, and in FIG. 19B the viewing angle has changed to buccal 190. In FIG. 19C, the marker 151 is different from FIGS. 19A and 19B, and next to the 3D model 29 at least three composed scan information (20A, 20B, 20C) and/or hybrid scan information (120A, 120B, 120C) are displayed of the marked area 151 on the 3D model 29 from different angles captured by the handheld intraoral scanning device. In this example, the one or more processors 11 is configured to select a plurality of composed scan information (20A, 20B, 20C) or hybrid scan information (120A, 120B, 120C) based on a position of the marker 151.

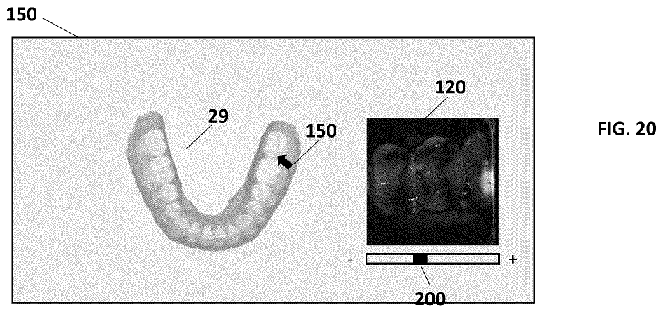

FIG. 20 illustrates another example of the displaying unit 150. In this example, a hybrid scan information 120 is displayed next to the 3D model 29, and where the plurality of composed scan information (20A,20B, 20C) in the hybrid scan information 120 is determined based on a position of the marker 150 on the 3D model 29. The one or more processors 11 is configured to add or remove composed scan information to and from the hybrid scan information 120 based on the position of the slider 200.

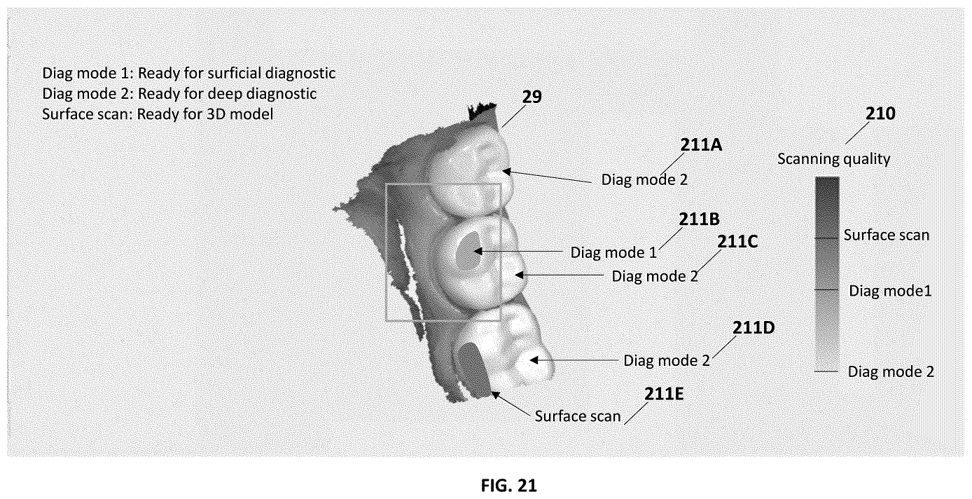

FIG. 21 illustrates an example of the displaying unit 150 during a scanning session. In this example, the user is able to perform a scanning of a dental arch 2 while a 3D model 29 is being generated or updated in real time, and while the 3D model is being generated or updated different colours and/or text markers may be added to areas on the 3D model. The colours and/or the text markers of a specific area on the 3D model indicate the quality of the captured visible light information and/or the internal light information for different purposes, such as for generating/updating the 3D model, surficial diagnostic (i.e. plaque), and deep diagnostic (i.e. cracks, carious lesion etc.). The scanning quality is depended on the amount of information (22, 24) being captured by the image sensor unit 7 or the consistency of the scanning. In this specific example, the displaying unit 150 includes a scanning quality bar 210 which includes colours that indicate the quality of the scanning for different purposes. In another example, the bar 210 may not be displayed, but instead, the quality is indicated on the 3D model directly either by colour and/or text marking (211A, 211B, 211C, 211D, 211E). A specific area 211E marked by a first colour and a text marking “Surface scan” indicates that the amount of visible light information received is just enough to generate/update the 3D model such that it reflects the area of the dental arch 2 of the patient. Another specific area 211B of the 3D model is marked with a different colour and text marking “Diag. mode 1” indicating that the amount of visible light information and the internal light information captured by the image sensor unit (7,7B) are just enough to generate/update the 3D model and to perform reliable surficial diagnostics of the another specific area. The remaining areas (211A, 211C, 211D) of the 3D model 29 are marked with yet another different colour and text marking “Diag. mode 2” indicating that the amount of visible light information and the internal light information captured by the image sensor unit (7,7B) are enough to generate/update the 3D model, to perform surficial diagnostic and to perform deep diagnostic. The diagnostic may be performed by the user or by the one or more processors 11 based on the composed scan information 20 or a plurality of composed scan information (20A, 20B, 20C) and a trained neural network.

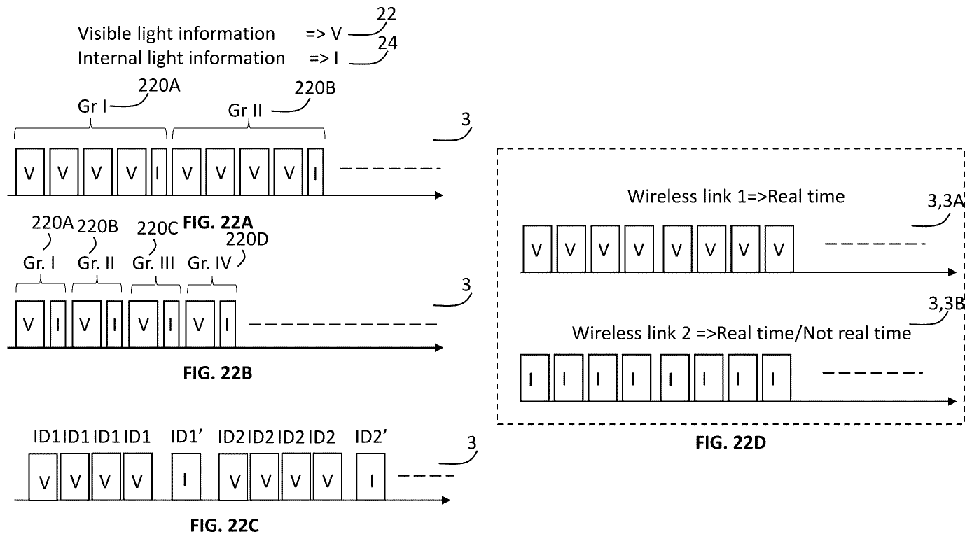

FIGS. 22A and 22B illustrate examples on how to wireless transmit the visible light information 22 and the internal light information 24 via a wireless communication interface 3 of the system 1. The wireless communication interface 3 may be between the handheld intraoral scanning device 10 and a processor 11 or a group of processors 11 of the one or more processors 11. In FIG. 22A the visible light information 22 and the internal light information 24 are being transmitted in groups (220A, 220B), and each of the groups (220A, 220B) includes multiple visible light information 22 data pulses (V) and one internal light information 24 data pulse (I) to be transmitted. In each of the groups (220A, 220B) the data pulses to be transmitted corresponds to the same area of the dental arch 2. FIG. 22B illustrates a similar example as in FIG. 22A, however, the number of visible light information 22 data pulses (V) are equal to the amount of internal light information 24 data pulses (I) to be transmitted. In FIG. 22C, the visible light information 22 pulse data (V) and internal light information 24 data pulses (I) that correspond to the same area of the dental arch 2 includes the same identification. In this example, the wireless transmission 3 of the visible light information 22 data pulses (V) includes a first identification (ID1) and wireless transmission 3 of the internal light information 24 data pulses (I) includes a second identification (ID1′), and wherein the composed scan information 20 includes the composition of the internal structure information 21B determined by the internal light information 24 data pulse (I) including the second identification (ID1′) and the visible light information 22 data pulse (V) including the first identification (ID1), and wherein the first identification (ID1) matches the second identification (ID1′). In FIG. 22D illustrates another example of the wireless communication interface 6. In this example, the visible light information 22 pulse data (V) are transmitted in real time in a first wireless link 3A of the wireless communication interface 3, and the internal light information 24 pulse data (I) are transmitted in a second wireless link 3B of the wireless communication interface 3. The first wireless link 3A and the second wireless link 3B may be two separate frequency channel of a wireless communication protocol, such as WIFI, Bluetooth, Bluetooth Low Energy or a 60 GHz wireless link. In another example, the first wireless link 3A and the second wireless link 3B may be two different communication protocols, such as the first wireless link 3A is based on WIFI and the second wireless link 3B is based on Bluetooth or Bluetooth Low energy. The second wireless link 3B may either transmit the pulse data (I) in real time or not real time depending on a scan mode of the system 1.

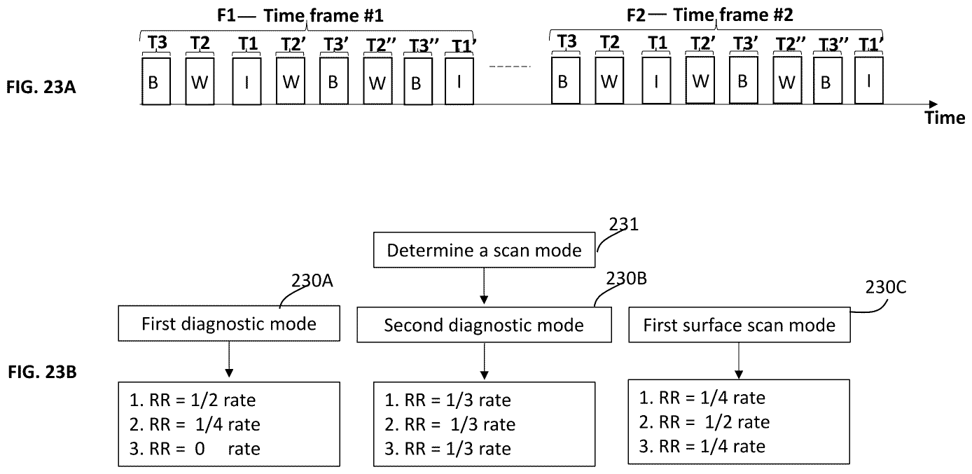

The handheld intraoral scanning device 10 is configured to emit light with different wavelengths during different time periods. The different time periods are grouped into the different wavelengths, such as a first group of the time periods and a second group of the time periods, where the first group includes emitted light at a near-infrared wavelength and the second group includes emitted light at a visible wavelength. The groups are arranged accordingly in a time frame. FIG. 23A illustrates a sequence of scans performed by the handheld intraoral scanning device 10, and this example, the scanning sequence includes two time-frames (F1, F2) where each of them includes a first group (T1, T1′), a second group (T2, T2′, T2″) and a third group (T3, T3′, T3″) of time periods. In this example, the first group includes a near-infrared wavelength, the second group includes a white-coloured wavelengths and the third group includes blue-coloured wavelength. Both time-frames includes the same sequence of different wavelengths, but, in another example, the sequence of the different wavelengths may chance according to a preference of the user or based on a quality of the 3D model being generated or updated. For example, the scanner 10 is scanning an area where the quality is ok for generating the surface of a 3D model 29 but needs more information for the one or mor processor 11 or the user to be able to diagnose a dental condition of the patient's dental arch 2. In this example, the next time frame will then include more time periods of for example blue-coloured and near-infrared wavelengths than white-coloured wavelength. Each of the groups of time periods includes a repetition rate. For example, the time frame (F1, F2) includes a first repetition rate (1. RR) of time periods of the first group (T1, T1′) of time periods, a second repetition rate (2. RR) of time periods of the second group (T2, T2′, T2″) of time periods, and a third repetition rate (3. RR) of time periods of the third group (T3, T3′, T3″) of time periods. In FIG. 23B it is seen that the one or more processors 11 is configured to determine the repetition rates based on a scan mode. The scan mode may be determined 231 by the one or more processors 11 or by the user via a user input of the system 1. In this example, the one or more processors 11 or the user is able to select between a first diagnostic mode 230A, a second diagnostic mode 230B and a first surface scan mode 230C. The first diagnostic mode may be suitable for performing surficial diagnostic (i.e. plaque, restorations etc.), the second diagnostic mode may be suitable for performing deep diagnostic (i.e. cracks, carious lesion etc.) and the first surface scan mode may be suitable for generating surface information to a 3D model 29. In the first diagnostic mode 230A the first repetition rate (1.RR) is larger than then the second repetition rate (2. RR), and the third repetition rate (3. RR) is not relevant as only two different wavelengths are used. In the second diagnostic mode 230B, all three repetition rates are the same (1. RR, 2. RR, 3. RR), and in the first surface scan mode 230C the first repetition rate (1. RR) is smaller than the second repetition rate (2. RR). In another example, a second surface scan mode 230D (not shown) may be selectable, and in the second scan mode 230D, the first and the third repetition rate is zero and only the second repetition rate is present, i.e. set to a level above zero.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

### Items

1. An intraoral scanning system configured to provide composed scan information, the system includes a handheld intraoral scanning device that includes:
a projector unit configured to emit light with different wavelengths during time periods onto at least a dental arch, wherein the different wavelengths include a near-infrared wavelength and a visible wavelength,
an image sensor unit configured to capture visible light information and internal light information from at least the dental arch caused by the visible wavelength and the near-infrared wavelength, respectively, and
   wherein the system further comprises one or more processors operably connected to the image sensor unit, the one or more processors is configured to:
receive the visible light information and the internal light information,
determine, in real time, surface information from the visible light information for generating or updating a three-dimensional (3D) model of the dental arch using the surface information,
determine internal structure information of the dental arch from the internal light information, and
determine composed scan information including a composition of the internal structure information and the visible light information.

2. The intraoral scanning system according to item 1, wherein the one or more processors is configured to determine fluorescence information of the dental arch from the visible light information.

3. The intraoral scanning system according to item 1 or 2, wherein the composed scan information includes a difference between the internal structure information and the visible light information, or between the internal structure information and the fluorescence information.

4. The intraoral scanning system according to item 2, wherein the fluorescence information includes green fluorescence information and/or red fluorescence information.

5. The intraoral scanning system according to item 4, wherein the one or more processors is configured to determine a first difference between the internal structure information and the green fluorescence information and a second difference between the internal structure information and the red fluorescence information, and wherein the composed scan information includes a summation of the first difference and the second difference.

6. The intraoral scanning system according to item 4, wherein the composed scan information includes a summation of the internal structure information, the green fluorescence information and the red fluorescence information.

7. The intraoral scanning system according to any of items 1 or 2, wherein the composed scan information includes a difference between the internal structure information and the surface information, or vice versa.

8. The intraoral scanning system according to any of the previous items, wherein the composition corresponds to a light intensity difference between light intensity of the internal structure information and light intensity of the visible light information, or vice versa, or a light intensity summation between light intensity of the internal structure information and light intensity of the visible light information.

9. The intraoral scanning system according to any of the previous items, wherein the internal structure information and the visible light information are weighted in the composed scan information, and wherein the internal structure information is weighted by a first weighting coefficient and the visible light information is weighted by a second weighting coefficient.

10. The intraoral scanning system according to item 9, wherein the one or more processors is configured to change the first and second weighting coefficient, and wherein the change corresponds to a change in brightness, contrast and/or transparency of the internal structure information and the visible light information.

10B. The intraoral scanning system according to items 9 or 10, wherein the visible light information includes both surface information and fluorescence information, the surface information is weighted by a first primary weighting coefficient, and the fluorescence information is weighted by a first secondary weighting coefficient.

11. The intraoral scanning system according to any of the previous items, wherein the one or more processors is configured to determine a plurality of composed scan information including the composed scan information and a second composed scan information, wherein the composed scan information is different from the second composed scan information.

12. The intraoral scanning system according to item 11, wherein each of the plurality of composed scan information includes different enhanced internal structures, and wherein the one or more processors is configured to label each of the plurality of composed scan information, and the label indicates the enhanced internal structure and/or the enhanced textural information of a composed scan information of the plurality of composed scan information.

13. The intraoral scanning system according to item 12, wherein the enhanced internal structure corresponds to a dental condition in a patient's teeth of the dental arch, wherein the dental condition corresponds to a crack, caries lesion, dentin-enamel junction, voids, plaque, and restorations.

14. The intraoral scanning system according to any of the previous items, wherein the projector unit includes one or more first light sources and at least one or more second light sources, wherein the one or more first light sources is configured to emit light within a first group of time periods at the near-infrared wavelength, and the one or more second light sources is configured to emit light within a second group of time periods at the visible wavelength, and wherein the visible light information include information corresponding to surface reflection of the dental arch caused by a first visible wavelength and captured by the image sensor unit, or, the visible light information include information corresponding to fluorescence information of the dental arch caused by the second visible wavelength and captured by the image sensor unit.

15. The intraoral scanning system according to any of the previous items, wherein the projector unit includes one or more first light sources, at least one or more second light sources, and at least one or more third light sources, wherein the one or more first light sources is configured to emit light within a first group of time periods at the near-infrared wavelength, the one or more second light sources is configured to emit light within a second group of time periods at a first visible wavelength, and the one or more third light sources is configured to emit light within a third group of time periods at a second visible wavelength, and wherein the first visible wavelength is different from the second visible wavelength, and wherein the visible light information includes information corresponding to surface reflections of the dental arch caused by the first visible wavelength and captured by the image sensor unit, and, the visible light information includes information corresponding to fluorescence information of the dental arch caused by the second visible wavelength and captured by the image sensor unit.

16. The intraoral scanning system according to item 14 and/or 15, wherein the one or more processors is configured to determine the surface information provided by the surface reflection of the first visible wavelength, and the composed scan information includes a composition of the internal structure information and the visible light information including the surface reflection of the first visible wavelength, and/or, the composed scan information includes a composition of the internal structure information and the visible light information including the fluorescence information of the second visible wavelength.

17. The intraoral scanning system according to any of the previous items, wherein the near-infrared wavelength is between 701 nm and 2500 nm, a first visible wavelength is between 380 nm and 700 nm, a second visible wavelength is between 100 nm and 500 nm, and the visible wavelength is between 100 nm and 700 nm.

18. The intraoral scanning system according to any of the previous items, wherein the projector unit is configured to emit light with different near-infrared wavelengths, and the image sensor unit is configured to capture internal light information from at least the dental arch caused by the different near-infrared wavelengths, and the one or more processors is configured to determine:
a plurality of internal structure information based on the captured internal light information caused by the different near-infrared wavelengths, and
a plurality of composed scan information, wherein each of the plurality of composed scan information is determined based on each of the plurality of internal structure information and the visible light information.

19. The intraoral scanning system according to item 18, comprising a memory unit that includes a relation between near-infrared wavelengths and penetration depth in teeth, and wherein the one or more processors is configured to determine a longest near-infrared wavelength of the different near-infrared wavelengths where an enhanced internal structure was identified by the one or more processors in one of the plurality of composed scan information, and wherein the one or more processors is configured to determine an internal depth information of the enhanced internal structure based on the longest near-infrared wavelength and the stored relation between near-infrared wavelengths and penetration depth of teeth.

20. The intraoral scanning system according to item 18, wherein the one or more processors is configured to determine depth information of an enhanced internal structure of a tooth or teeth of the dental arch, wherein the determined depth information is based on the plurality of composed scan information that is determined based on the plurality of internal structure information and the visible light information.

21. The intraoral scanning system according to item 19, wherein the internal depth information is determined based on a trained neural network, wherein the trained neural network is configured to receive the plurality of composed scan information and the visible light information and estimate one or more sub internal depth information between a first depth information and a second depth information, wherein the first depth information and the second depth information are determined by a first composed scan information and a second composed scan information of the plurality of composed scan information, respectively.

22. The intraoral scanning system according to item 21, wherein the trained neural network is trained based on a training data set that includes multiple composed scan information and visible light information of other patients' dental arch.

23. The intraoral scanning system according to item 22, wherein the multiple composed scan information includes handheld intraoral scan information, X-ray scan information, OCT scan information of the same patients' dental arch.

24. The intraoral scanning system according to any of items 21 to 23, wherein trained neural network includes one or more of following topologies, Convolutional Neural Networks (CNN), Three-circle model, persistent homology method and algebraic topology.

25. The intraoral scanning system according to any of the previous items, wherein the image sensor unit includes at least one image sensor configured to capture light from within a range of between 100 nm and 2500 nm, or the image sensor unit includes a plurality of image sensors, where each of the plurality of image sensors is configured to capture light between 701 nm and 2500 nm, 100 nm and 500 nm, or 380 nm and 700 nm.

26. The intraoral scanning system according to any of the items, wherein the internal structure information is determined based on multiple captured internal light information, wherein the multiple captured internal light information is captured by the image sensor unit at different angles between the dental arch and the image sensor unit.

27. The intraoral scanning system according to item 26, wherein the internal structure information is determined based on an average or a weighted average of the multiple captured internal light information.

28. The intraoral scanning system according to item 2, wherein the one or more processors is configured to determine the surface information and/or the fluorescence information based on multiple captured visible light information, wherein the multiple captured visible light information is captured by the image sensor unit at different angles between the dental arch and the image sensor unit.

29. The intraoral scanning system according to item 28, wherein the determined surface information and/or the fluorescence information is based on an average or a weighted average of the multiple captured visible light information.

30. The intraoral scanning system according to item 11, wherein the one or more processors is configured to determine hybrid scan information including the plurality of composed scan information.

31. The intraoral scanning system according to item 30, wherein the hybrid scan information includes a plurality of channels, wherein each of the plurality of channels corresponds to each of the plurality of composed scan information.

32. The intraoral scanning system according to any of items 30 and 31, wherein the one or more processors is configured to weight differently each of the plurality of channels with a channel weighting coefficient.

33. The intraoral scanning system according to item 32, wherein a change of the channel weighting coefficient corresponds to a change in brightness, contrast and/or transparency of a composed scan information corresponding to a channel of the plurality of channels.

34. The intraoral scanning system according to any of items 31 to 33, wherein each of the plurality of channels is assigned to a colour.

35. The intraoral scanning system according to any of items 31 to 34, wherein the plurality of channels includes a first channel that corresponds to a background information that includes a first composed scan information of the plurality of composed scan information, and a second channel that corresponds to a first stacked information that includes a second composed scan information of the plurality of composed scan information, and wherein the first stacked information is aligned with and stacked on the background information.

36. The intraoral scanning system according to any of items 31 to 35, wherein each of the plurality of channels includes a single composed scan information of the plurality of composed scan information or a combination of composed scan information of the plurality of composed scan information, and wherein the combined composed scan information includes a combination of at least two composed scan information of the plurality of composed scan information.

37. The intraoral scanning system according to item 36, wherein the combination of at least two composed scan information includes a summation of a first composed scan information of the plurality of scan information and a second composed scan information of the plurality of scan information.

38. The intraoral scanning system according to item 37, wherein the combination of the at least two composed scan information includes a difference between a first composed scan information of the plurality of scan information and a second composed scan information of the plurality of scan information.

39. The intraoral scanning system according to any of the previous items, wherein the one or more processors is configured to display the composed scan information on a displaying unit of the intraoral scanning system.

40. The intraoral scanning system according to any of the previous items, wherein the one or more processors is configured to determine an enhanced internal structure and/or enhanced texture information based on the composed scan information.

41. The intraoral scanning system according to item 40, wherein the one or more processors is configured to apply enhanced internal structure and/or the enhanced texture information onto the 3D model or a 2D surface image of a tooth or teeth of the dental arch.

42. The intraoral scanning system according to any of items 19 to 24, and item 41, wherein the one or more processors is configured to display on a displaying unit of the system the internal depth information of the corresponding enhanced internal structure and/or the enhanced texture information.

43. The intraoral scanning system according to item 42, wherein the one or more processor is configured to display on the displaying unit the internal depth information on the 3D model, on the 2D surface image, next to the 3D model, or next to the 2D surface.

44. The intraoral scanning system according to item 11, wherein the one or more processors is configured to:
display the three-dimensional (3D) model of the dental arch on a displaying unit of the intraoral scanning system;
display a marker over a portion of the 3D model of the dental arch on the displaying unit;
change a relative position between the marker and the 3D model of the dental arch based on input from a user input device of the intraoral scanning system;
identify, from both the 3D model of the dental arch and the plurality of composed scan information, wherein each of the plurality of composed scan information includes internal light information and visible light information captured at an angle and position, a composed scan information of the plurality of composed scan information that approximates a relative angle and position between the marker relative to the 3D model of the dental arch; and
display the identified composed scan information determined for the angle and position that approximates the angle and position between the marker relative to the 3D model of the dental arch.

45. The intraoral scanning system according to item 11, wherein the one or more processors is configured to:
display the three-dimensional (3D) model of the dental arch on a displaying unit of the intraoral scanning system;
display a marker (a viewing marker, a cursor) over a portion of the 3D model of the dental arch on a displaying unit of the system;
continuously, as the user changes the relative position between the marker and the 3D model of the patient's dental arch based on a user input:
   identify, from both the 3D model of the dental arch and the plurality of composed scan information, wherein the plurality of composed scan information includes a group of composed scan information determined for internal light information and visible light information taken at the same angle and position relative to the dental arch, a group of composed scan information of the plurality of composed scan information determined at an angle and position that approximate the angle and position of the marker relative to the displayed 3D model of the dental arch; and
   display on the displaying unit the at least one of the group of composed scan information corresponding to the angle and position that approximate the angle and position of the marker relative to the displayed 3D model of the dental arch.

46. The intraoral scanning system according to item 11, wherein the one or more processors is configured to:
receive, based on a user input, one or more of the plurality of composed scan information including an internal structure;
correlate the one or more of the plurality of composed scan information to the 3D model to associate a location of the one or more of the plurality of composed scan information with a corresponding location in the 3D model; and
display the 3D model on a displaying unit of the system showing a section through the 3D model with the one or more of the plurality of composed scan information included on the section, showing internal structures including the internal structure.

47. The intraoral scanning system according to item 12, wherein the one or more processors is configured to:
display, in a user interface of the intraoral scanning system, a label selector that is configured to select one or more of the labelled plurality of composed scan information, based on a user input,
select, based on a user input, one or more areas-of-interest on the 3D model, and where each of the area-of-interest corresponds to a location of a tooth or teeth of the dental arch on the 3D model,
select one or more composed scan information based on the selected one or more area-of-interest on the 3D model and the one or more selected labels, and
display the one or more composed scan information on a displaying unit of the intraoral scanning system.

48. The intraoral scanning system according to item 2 and any of items 39 to 47, wherein the displaying of the composed scan information includes combination of the composed scan information and the surface information or the fluorescence information.

49. The intraoral scanning system according to item 48, wherein the composed scan information, the surface information or the fluorescence information correspond to the same tooth or teeth of the dental arch.

50. The intraoral scanning system according to any of items 48 and 49, wherein the surface information or the fluorescence information relating to a surface of the tooth or teeth of the dental arch is coloured with one or more colours, and the composed scan information is coloured with one or more colours not relating to the one or more colours of the surface.

51. The intraoral scanning system according to item 50, wherein the one or more processors is configured to select the one or more colours of the surface and the composed scan information based on a user preference or based on a colour machine learning output of a colour machine learning unit of the system that is trained based on a training data set including user preference of multiple users of the system.

52. The intraoral scanning system according to any of items 30 to 35, wherein the hybrid scan information is configured to be displayed on a displaying unit of the intraoral scanning system.

53. The intraoral scanning system according to item 52, wherein the displayed hybrid scan information includes a combination of the hybrid scan information and the surface information or fluorescence information.

54. The intraoral scanning system according to item 52 or 53, wherein the one or more processors is configured to select, based on a user input, the plurality of the composed scan information of the hybrid scan information to be displayed.

55. The intraoral scanning system according to any of items 52 to 54, wherein the one or more processors is configured to, based on a user input, add or remove one or more composed scan information to the plurality of composed scan information of the hybrid scan information.

56. The intraoral scanning system according to any of items 52 to 55, wherein the displayed hybrid scan information includes one or more colour representing the surface information or the fluorescence information, and the hybrid scan information is represented by one or more colours not relating to the one or more colours representing the surface information or the fluorescence information.

57. The intraoral scanning system according to item 56, wherein the one or more processors is configured to, based on a user input, change the colouring of the one or more colours of the hybrid scan information and the one or more colour of the surface information or the fluorescence information.

58. The intraoral scanning system according to any of items 52 to 57, wherein the one or more processors is configured to:
display the three-dimensional (3D) model of the dental arch on a displaying unit of the intraoral scanning system;
display a marker (a viewing marker, a cursor) over a portion of the 3D model of the dental arch on the displaying unit;
change a relative position between the marker and the 3D model of the dental arch based on input from a user input device of the intraoral scanning system;
identify, from both the 3D model of the dental arch and the plurality of composed scan information, wherein each of the plurality of composed scan information includes internal light information and visible light information captured at an angle and position, two or more composed scan information that approximates a relative angle and position between the marker relative to the 3D model of the dental arch; and
display the hybrid scan information including the identified two or more composed scan information, wherein the internal light information and the visible light information of the selected two or more composed scan information correspond to the angle and position that approximates the angle and position between the marker relative to the 3D model of the dental arch.

59. The intraoral scanning system according to any of items 52 to 57, wherein the one or more processors is configured to:
display the hybrid scan information including the plurality of composed scan information on a displaying unit of the system;
display a slider (a viewing marker, a cursor) next to the displayed hybrid scan information, on the displaying unit;
change a position of the slider based on a user input from a user input device of the intraoral scanning system; and
add or remove a composed scan information to the hybrid scan information based on the position of the slider.

60. The intraoral scanning system according to any of the previous items, wherein the handheld intraoral scanning device is configured to emit light with different wavelengths during different time periods.

61. The intraoral scanning system according to items 14, wherein the first group of time periods and the second group of time periods are arranged accordingly in a time frame.

62. The intraoral scanning system according to item 61, wherein the time frame includes a first repetition rate of time periods of the first group of time periods and a second repetition rate of time periods of the second group of time periods.

63. The intraoral scanning system according to items 15 and 61, wherein the time frame includes a third repetition rate of time periods of the third group of time periods.

64. The intraoral scanning system according to items 62 and/or 63, wherein the first repetition rate, the second repetition rate and/or the third repetition rate are determined by the one or more processors based on a scan mode, wherein the scan mode is determined based on a user input.

65. The intraoral scanning system according to item 64, wherein the scan mode is one of following modes:
a first diagnostic mode where the first repetition rate is larger or equal than the second repetition rate;
a second diagnostic mode where the first repetition rate and the third repetition rate are larger than or equal the second repetition rate; and
a first surface scan mode where the first repetition rate is smaller than the second repetition rate.

66. The intraoral scanning system according to item 65, wherein the one or more processors is configured to, based on a user input, toggle between different scan modes during a scanning session of the dental arch based on a user input.

67. The intraoral scanning system according to any of the previous items, comprising a wireless communication interface between the handheld intraoral scanning device and a processor of the one or more processors or a group of processors of the one or more processors, and wherein the visible light information is being wireless transmitted with a first data rate, and the internal light information is being wireless transmitted with a second data rate.

68. The intraoral scanning system according to item 67, wherein the one or more processors is configured to change the first data rate and/or the second data rate according to a scan mode.

69. The intraoral scanning system according to any of items 67 and 68, wherein the first data rate is different from the second data rate.

70. The intraoral scanning system according to any of items 1 to 68, wherein the visible light information and the internal light information are wireless transmitted at the same data rate.

71. The intraoral scanning system according to any of the previous items, wherein wireless transmission of the visible light information and the internal light information are being wireless transmitted in groups, wherein a group includes visible light information and corresponding internal light information.

72. The intraoral scanning system according to any of the previous items, wherein wireless transmission of the visible light information includes a first identification and wireless transmission of the internal light information includes a second identification, and wherein the composed scan information includes the composition of the internal structure information determined by the internal light information including the second identification and the visible light information including the first identification, and wherein the first identification matches the second identification.

73. The intraoral scanning system according to any of the previous items, wherein the visible light information and the internal light information are wireless transmitted asynchronously to a processor or a group of processors of the one or more processors.

74. The intraoral scanning system according to any of items 1 to 72, wherein the visible light information and the internal light information are wireless transmitted synchronously to a processor or a group of processors of the one or more processors.

75. The intraoral scanning system according to any of the previous items, wherein the one or more processors is configured to select one or more modulation schemes for modulating the visible light information and/or the internal light information to be transmitted over a wireless network of the intraoral scanning system.

76. The intraoral scanning system according to item 75, wherein the one or more processors is configured to select one or more modulation schemes based on a scan mode or information type to be transmitted over the wireless network.

77. The intraoral scanning system according to any of items 75 and 76, wherein the one or more modulation schemes include following modulation schemes:
MOK (M-ary Orthogonal Keying),
ACM (Automatic Coding and Modulation Scheme),
MBOK (M-ary Bi-Orthogonal Keying),
CCK (Complementary Code Keying),
CCSK (Cyclic Code Shift Keying),
PSK (Phase Shift Keying),
PPM (Pulse Position Modulation),
QAM (Quadrature Amplitude Modulation),
OCDM (Orthogonal Chirp Division Multiplexing), and
OFDM (Orthogonal Frequency-Division Multiplexing).

78. The intraoral scanning system according to any of the previous items, comprising a user interface configured to receive a user input.

1A. An intraoral scanning system configured to provide composed scan information, the system includes a handheld intraoral scanning device that includes:
a projector unit configured to emit light at a near-infrared wavelength, a blue-coloured wavelength and white-coloured wavelengths onto at least a dental arch,
an image sensor unit configured to capture visible light information and internal light information from at least the dental arch caused by the emitted light of the projector unit, and
   wherein the system further comprises one or more processors operably connected to the image sensor unit, the one or more processors is configured to:
receive the visible light information that relates to the white-coloured wavelengths and the blue-coloured wavelength and the internal light information that relates to the near-infrared wavelength,
determine, in real time, surface information from the visible light information for generating or updating a three-dimensional (3D) model of the dental arch using the surface information,
determine fluorescence information of the dental arch from the visible light information that relates to the blue-wavelength,
determine internal structure information of the dental arch from the internal light information,
determine composed scan information including a composition of the internal structure information and the fluorescence information.

2A. The intraoral scanning system according to item 1, wherein the fluorescence information includes at least a green fluorescence information.

3A. The intraoral scanning system according to item 2A, wherein the fluorescence information includes at least a red fluorescence information.

4A. The intraoral scanning system according to any of the items, wherein the composed scan information includes a difference between the internal structure information and the fluorescence information or vice versa.

5A. The intraoral scanning system according to any of the items, wherein the composed scan information includes a summation of the internal structure information and the fluorescence information.

6A. The intraoral scanning system according to items 2 and 3, wherein the one or more processors is configured to determine a first difference between the internal structure information and the green fluorescence information and a second difference between the internal structure information and the red fluorescence information, and wherein the composed scan information includes a summation of the first difference and the second difference.

7A. The intraoral scanning system according to items 2 and 3, wherein the composed scan information includes a summation of the internal structure information, the green fluorescence information and the red fluorescence information.

### Items

1B. An intraoral scanning system configured to provide composed scan information, the system includes a handheld intraoral scanning device that includes:
a projector unit configured to emit light at a near-infrared wavelength and white-coloured wavelengths onto at least a dental arch,
an image sensor unit configured to capture visible light information and internal light information from at least the dental arch caused by the emitted light of the projector unit, and
   wherein the system further comprises one or more processors operably connected to the image sensor unit, the one or more processors is configured to:
receive the visible light information and the internal light information,
determine, in real time, surface information from the visible light information for generating or updating a three-dimensional (3D) model of the dental arch using the surface information,
determine internal structure information of the dental arch from the internal light information, and
determine composed scan information including a composition of the internal structure information and the surface information.

2B. The intraoral scanning system according to item 1B, wherein the composed scan information includes a difference between the internal structure information and the surface information or vice versa.

3B. The intraoral scanning system according to any of the previous items, wherein the one or more processors is configured to determine a first type of restorations based on the composed scan information including the difference between the internal structure information and the surface information, and a second type of restorations based on the composed scan information including the difference between the surface information and the internal structure information.

4B. The intraoral scanning system according to item 3B, wherein the one or more processors is configured to determine a plurality of composed scan information comprising at least the composed scan information that includes the difference between the internal structure information and the surface information and a second composed scan information including the difference between the surface information and the internal structure information.

1. An intraoral scanning system configured to provide composed scan information, the system includes a handheld intraoral scanning device that includes:
a projector unit configured to emit light with different wavelengths during time periods onto at least a dental arch, wherein the different wavelengths include a near-infrared wavelength and a visible wavelength,
an image sensor unit configured to capture visible light information and internal light information from at least the dental arch caused by the visible wavelength and the near-infrared wavelength, respectively, and
wherein the system further comprises one or more processors operably connected to the image sensor unit, the one or more processors is configured to:
receive the visible light information and the internal light information,
determine, in real time, surface information from the visible light information for generating or updating a three-dimensional (3D) model of the dental arch using the surface information,
determine internal structure information of the dental arch from the internal light information, and
determine composed scan information including a composition of the internal structure information and the visible light information, wherein the composition includes a light intensity difference between the internal structure information and the visible light information.

2. The intraoral scanning system according to claim 1, wherein the one or more processors is configured to determine fluorescence information of the dental arch from the visible light information.

3. The intraoral scanning system according to claim 1, wherein the composed scan information includes a difference between the internal structure information and the visible light information, or between the internal structure information and the fluorescence information.

4. The intraoral scanning system according to claim 2, wherein the fluorescence information includes green fluorescence information and/or red fluorescence information.

5. The intraoral scanning system according to claim 4, wherein the one or more processors is configured to determine a first difference between the internal structure information and the green fluorescence information and a second difference between the internal structure information and the red fluorescence information, and wherein the composed scan information includes a summation of the first difference and the second difference.

6. The intraoral scanning system according to claim 4, wherein the composed scan information includes a summation of the internal structure information, the green fluorescence information and the red fluorescence information.

7. The intraoral scanning system according to claim 1, wherein the composed scan information includes a difference between the internal structure information and the surface information, or vice versa.

8. The intraoral scanning system according claim 1, wherein the composition corresponds to a light intensity difference between light intensity of the internal structure information and light intensity of the visible light information, or vice versa, or a light intensity summation between light intensity of the internal structure information and light intensity of the visible light information.

9. The intraoral scanning system according to claim 1, wherein the internal structure information and the visible light information are weighted in the composed scan information, and wherein the internal structure information is weighted by a first weighting coefficient and the visible light information is weighted by a second weighting coefficient.

10. The intraoral scanning system according to claim 9, wherein the one or more processors is configured to change the first and second weighting coefficient, and wherein the change corresponds to a change in brightness, contrast and/or transparency of the internal structure information and the visible light information.

11. (canceled)

12. The intraoral scanning system according to claim 1, wherein the one or more processors is configured to determine a plurality of composed scan information including the composed scan information and a second composed scan information, wherein the composed scan information is different from the second composed scan information.

13. The intraoral scanning system according to claim 12, wherein each of the plurality of composed scan information includes different enhanced internal structures, and wherein the one or more processors is configured to label each of the plurality of composed scan information, and the label indicates the enhanced internal structure and/or the enhanced textural information of a composed scan information of the plurality of composed scan information.

14. The intraoral scanning system according to claim 13, wherein the enhanced internal structure corresponds to a dental condition in a patient's teeth of the dental arch, wherein the dental condition corresponds to a crack, caries lesion, dentin-enamel junction, voids, plaque, and restorations.

15. The intraoral scanning system according to claim 1, wherein the projector unit includes one or more first light sources and at least one or more second light sources, wherein the one or more first light sources is configured to emit light within a first group of time periods at the near-infrared wavelength, and the one or more second light sources is configured to emit light within a second group of time periods at the visible wavelength, and wherein the visible light information include information corresponding to surface reflection of the dental arch caused by a first visible wavelength and captured by the image sensor unit, or, the visible light information include information corresponding to fluorescence information of the dental arch caused by a second visible wavelength and captured by the image sensor unit.

16. The intraoral scanning system according to claim 1, wherein the projector unit includes one or more first light sources, at least one or more second light sources, and at least one or more third light sources, wherein the one or more first light sources is configured to emit light within a first group of time periods at the near-infrared wavelength, the one or more second light sources is configured to emit light within a second group of time periods at a first visible wavelength, and the one or more third light sources is configured to emit light within a third group of time periods at a second visible wavelength, and wherein the first visible wavelength is different from the second visible wavelength, and wherein the visible light information includes information corresponding to surface reflections of the dental arch caused by the first visible wavelength and captured by the image sensor unit, and, the visible light information includes information corresponding to fluorescence information of the dental arch caused by the second visible wavelength and captured by the image sensor unit.

17. The intraoral scanning system according to claim 15, wherein the one or more processors is configured to determine the surface information provided by the surface reflection of the first visible wavelength, and the composed scan information includes a composition of the internal structure information and the visible light information including the surface reflection of the first visible wavelength, and/or, the composed scan information includes a composition of the internal structure information and the visible light information including the fluorescence information of the second visible wavelength.

18. An intraoral scanning system configured to provide composed scan information, the system includes a handheld intraoral scanning device that includes:
a projector unit configured to emit light with different wavelengths during time periods onto at least a dental arch, wherein the different wavelengths include a near-infrared wavelength and a visible wavelength,
an image sensor unit configured to capture visible light information and internal light information from at least the dental arch caused by the visible wavelength and the near-infrared wavelength, respectively, and
wherein the system further comprises one or more processors operably connected to the image sensor unit, the one or more processors is configured to:
receive the visible light information and the internal light information,
determine, in real time, surface information from the visible light information for generating or updating a three-dimensional (3D) model of the dental arch using the surface information,
determine internal structure information of the dental arch from the internal light information, and
determine composed scan information including a combination of intensity levels of each pixel of the image sensor unit that relates to different wavelengths.
